SEQUENCE LISTING (90 sequences)

SEQ ID NO: 1 - DNA, 35 bp, synthetic construct
  source 1..35: mol_type = other DNA
tgtcggtctctggctccacaggtggctgccaatac

SEQ ID NO: 2 - DNA, 36 bp, synthetic construct
  source 1..36: mol_type = other DNA
cacgggagccactgactcccctgaagactcggatgt

SEQ ID NO: 3 - DNA, 15 bp, synthetic construct
  source 1..15: mol_type = other DNA
gcagcttcgggtcct

SEQ ID NO: 4 - DNA, 20 bp, synthetic construct
  source 1..20: mol_type = other DNA
ggataagtggaggagtgtct

SEQ ID NO: 5 - DNA, 18 bp, synthetic construct
  source 1..18: mol_type = other DNA
tcaacaagctccacggtg

SEQ ID NO: 6 - DNA, 17 bp, synthetic construct
  source 1..17: mol_type = other DNA
tccgcaacaactcagcc

SEQ ID NO: 7 - DNA, 18 bp, synthetic construct
  source 1..18: mol_type = other DNA
tcaacaagctccacggtg

SEQ ID NO: 8 - DNA, 17 bp, synthetic construct
  source 1..17: mol_type = other DNA
tccgcaacaactcagcc

SEQ ID NO: 9 - DNA, 51 bp, synthetic construct
  source 1..51: mol_type = other DNA
tcagcacacaaagccaaaaatttatttttctgtgcaaaggaaattaaggag

SEQ ID NO: 10 - DNA, 49 bp, synthetic construct
  source 1..49: mol_type = other DNA
tattggtggagctaaacttaaagccttttctgtacaatccctttgagtg

SEQ ID NO: 11 - DNA, 18 bp, synthetic construct
  source 1..18: mol_type = other DNA
cggtggacaaattgtcac

SEQ ID NO: 12 - DNA, 22 bp, synthetic construct
  source 1..22: mol_type = other DNA
cttctctggatttaacacactt

SEQ ID NO: 13 - DNA, 28 bp, synthetic construct
  source 1..28: mol_type = other DNA
ttacaagcttaaagaatgtctgaacact

SEQ ID NO: 14 - DNA, 27 bp, synthetic construct
  source 1..27: mol_type = other DNA
ttgaatttaggtgaaacatttgtcacg

SEQ ID NO: 15 - DNA, 28 bp, synthetic construct
  source 1..28: mol_type = other DNA
ttacaagcttaaagaatgtctgaacact

SEQ ID NO: 16 - DNA, 27 bp, synthetic construct
  source 1..27: mol_type = other DNA
ttgaatttaggtgaaacatttgtcacg

SEQ ID NO: 17 - DNA, 38 bp, synthetic construct
  source 1..38: mol_type = other DNA
gggggggattgaatttaggtgaaacatttgtcacgcac

SEQ ID NO: 18 - DNA, 38 bp, synthetic construct; identical to SEQ ID NO: 17
  source 1..38: mol_type = other DNA

SEQ ID NO: 19 - DNA, 25 bp, synthetic construct
  source 1..25: mol_type = other DNA
gtttcacctaaattcaatccccccc

SEQ ID NO: 20 - DNA, 27 bp, synthetic construct
  source 1..27: mol_type = other DNA
gggggggattgaatttaggtggccccg

SEQ ID NO: 21 - DNA, 19 bp, synthetic construct
  source 1..19: mol_type = other DNA
cggggccacctaaattcaa

SEQ ID NO: 22 - DNA, 38 bp, synthetic construct
  source 1..38: mol_type = other DNA
accccgtgcttgaatttaggtgaaacatttgtcacgca

SEQ ID NO: 23 - DNA, 38 bp, synthetic construct; identical to SEQ ID NO: 22
  source 1..38: mol_type = other DNA

SEQ ID NO: 24 - DNA, 25 bp, synthetic construct
  source 1..25: mol_type = other DNA
tttcacctaaattcaagcacggggt

SEQ ID NO: 25 - DNA, 28 bp, synthetic construct
  source 1..28: mol_type = other DNA
ccccgtgcttgaatttaggagggcctgg

SEQ ID NO: 26 - DNA, 20 bp, synthetic construct
  source 1..20: mol_type = other DNA
ccaggccctcctaaattcaa

SEQ ID NO: 27 - DNA, 37 bp, synthetic construct
  source 1..37: mol_type = other DNA
ttgaatttaggtgaaacatttgtcacgcacgtccacc

SEQ ID NO: 28 - DNA, 37 bp, synthetic construct; identical to SEQ ID NO: 27
  source 1..37: mol_type = other DNA

SEQ ID NO: 29 - DNA, 24 bp, synthetic construct
  source 1..24: mol_type = other DNA
ggtggacgtgcgtgacaaatgttt

SEQ ID NO: 30 - DNA, 22 bp, synthetic construct
  source 1..22: mol_type = other DNA
cccgtttgtcacgcacgtccac

SEQ ID NO: 31 - DNA, 16 bp, synthetic construct
  source 1..16: mol_type = other DNA
gtgcgtgacaaacggg

SEQ ID NO: 32 - DNA, 38 bp, synthetic construct
  source 1..38: mol_type = other DNA
ttgaatttaggtgaaacatttgtcacgcaaccgcgaat

SEQ ID NO: 33 - DNA, 38 bp, synthetic construct; identical to SEQ ID NO: 32
  source 1..38: mol_type = other DNA

SEQ ID NO: 34 - DNA, 25 bp, synthetic construct
  source 1..25: mol_type = other DNA
attcgcggttgcgtgacaaatgttt

SEQ ID NO: 35 - DNA, 24 bp, synthetic construct
  source 1..24: mol_type = other DNA
tcgcgttttgtcacgcaaccgcga

SEQ ID NO: 36 - DNA, 16 bp, synthetic construct
  source 1..16: mol_type = other DNA
gcgtgacaaaacgcga

SEQ ID NO: 37 - DNA, 39 bp, synthetic construct
  source 1..39: mol_type = other DNA
aggccgcttgttgaatttaggtgaaacatttgtcacgca

SEQ ID NO: 38 - DNA, 39 bp, synthetic construct; identical to SEQ ID NO: 37
  source 1..39: mol_type = other DNA

SEQ ID NO: 39 - DNA, 27 bp, synthetic construct
  source 1..27: mol_type = other DNA
gtttcacctaaattcaacaagcggcct

SEQ ID NO: 40 - DNA, 29 bp, synthetic construct
  source 1..29: mol_type = other DNA
ggccgcttgttgaatttaggtggaggccc

SEQ ID NO: 41 - DNA, 20 bp, synthetic construct
  source 1..20: mol_type = other DNA
gggcctccacctaaattcaa

SEQ ID NO: 42 - DNA, 40 bp, synthetic construct
  source 1..40: mol_type = other DNA
ttgaatttaggtgaaacatttgtcacgcacagcctctagt

SEQ ID NO: 43 - DNA, 40 bp, synthetic construct; identical to SEQ ID NO: 42
  source 1..40: mol_type = other DNA

SEQ ID NO: 44 - DNA, 26 bp, synthetic construct
  source 1..26: mol_type = other DNA
actagaggctgtgcgtgacaaatgtt

SEQ ID NO: 45 - DNA, 26 bp, synthetic construct
  source 1..26: mol_type = other DNA
tgcgtggtgtcacgcacagcctctag

SEQ ID NO: 46 - DNA, 16 bp, synthetic construct
  source 1..16: mol_type = other DNA
tgcgtgacaccacgca

SEQ ID NO: 47 - DNA, 38 bp, synthetic construct
  source 1..38: mol_type = other DNA
ttgaatttaggtgaaacatttgtcacgcgctctctata

SEQ ID NO: 48 - DNA, 38 bp, synthetic construct; identical to SEQ ID NO: 47
  source 1..38: mol_type = other DNA

SEQ ID NO: 49 - DNA, 24 bp, synthetic construct
  source 1..24: mol_type = other DNA
tatagagagcgcgtgacaaatgtt

SEQ ID NO: 50 - DNA, 21 bp, synthetic construct
  source 1..21: mol_type = other DNA
agcgcctgtcacgcgctctct

SEQ ID NO: 51 - DNA, 14 bp, synthetic construct
  source 1..14: mol_type = other DNA
gcgtgacaggcgct

SEQ ID NO: 52 - DNA, 38 bp, synthetic construct
  source 1..38: mol_type = other DNA
ttgaatttaggtgaaacatttgtcacgcactcacgact

SEQ ID NO: 53 - DNA, 38 bp, synthetic construct; identical to SEQ ID NO: 52
  source 1..38: mol_type = other DNA

SEQ ID NO: 54 - DNA, 24 bp, synthetic construct
  source 1..24: mol_type = other DNA
agtcgtgagtgcgtgacaaatgtt

SEQ ID NO: 55 - DNA, 20 bp, synthetic construct
  source 1..20: mol_type = other DNA
cgcggtgtcacgcactcacg

SEQ ID NO: 56 - DNA, 14 bp, synthetic construct
  source 1..14: mol_type = other DNA
tgcgtgacaccgcg

SEQ ID NO: 57 - DNA, 38 bp, synthetic construct
  source 1..38: mol_type = other DNA
ttacaagcttaaagaatgtctgaacactcgggtctatt

SEQ ID NO: 58 - DNA, 38 bp, synthetic construct; identical to SEQ ID NO: 57
  source 1..38: mol_type = other DNA

SEQ ID NO: 59 - DNA, 25 bp, synthetic construct
  source 1..25: mol_type = other DNA
aatagacccgagtgttcagacattc

SEQ ID NO: 60 - DNA, 27 bp, synthetic construct
  source 1..27: mol_type = other DNA
acgatgcgtctgaacactcgggtctat

SEQ ID NO: 61 - DNA, 18 bp, synthetic construct
  source 1..18: mol_type = other DNA
agtgttcagacgcatcgt

SEQ ID NO: 62 - DNA, 40 bp, synthetic construct
  source 1..40: mol_type = other DNA
acacggcgttacaagcttaaagaatgtctgaacactctcc

SEQ ID NO: 63 - DNA, 40 bp, synthetic construct; identical to SEQ ID NO: 62
  source 1..40: mol_type = other DNA

SEQ ID NO: 64 - DNA, 27 bp, synthetic construct
  source 1..27: mol_type = other DNA
acattctttaagcttgtaacgccgtgt

SEQ ID NO: 65 - DNA, 28 bp, synthetic construct
  source 1..28: mol_type = other DNA
acggcgttacaagcttaaaaaagcagat

SEQ ID NO: 66 - DNA, 22 bp, synthetic construct
  source 1..22: mol_type = other DNA
atctgcttttttaagcttgtaa

SEQ ID NO: 67 - DNA, 35 bp, synthetic construct
  source 1..35: mol_type = other DNA
taaatagccgggatccgcaacaactcagccatcca

SEQ ID NO: 68 - DNA, 35 bp, synthetic construct; identical to SEQ ID NO: 67
  source 1..35: mol_type = other DNA

SEQ ID NO: 69 - DNA, 23 bp, synthetic construct
  source 1..23: mol_type = other DNA
ttgttgcggatcccggctattta

SEQ ID NO: 70 - DNA, 23 bp, synthetic construct
  source 1..23: mol_type = other DNA
ttgttgcggatcccggctattta

SEQ ID NO: 71 - DNA, 25 bp, synthetic construct
  source 1..25: mol_type = other DNA
aaatagccgggatccgccggacgtg

SEQ ID NO: 72 - DNA, 16 bp, synthetic construct
  source 1..16: mol_type = other DNA
cacgtccggcggatcc

SEQ ID NO: 73 - DNA, 34 bp, synthetic construct
  source 1..34: mol_type = other DNA
ggatccgcaacaactcagccatcccagtccttat

SEQ ID NO: 74 - DNA, 34 bp, synthetic construct; identical to SEQ ID NO: 73
  source 1..34: mol_type = other DNA

SEQ ID NO: 75 - DNA, 22 bp, synthetic construct
  source 1..22: mol_type = other DNA
ataaggactgggatggctgagt

SEQ ID NO: 76 - DNA, 22 bp, synthetic construct
  source 1..22: mol_type = other DNA
ataaggactgggatggctgagt

SEQ ID NO: 77 - DNA, 25 bp, synthetic construct
  source 1..25: mol_type = other DNA
tcggcctcagccatcccagtcctta

SEQ ID NO: 78 - DNA, 16 bp, synthetic construct
  source 1..16: mol_type = other DNA
ggatggctgaggccga

SEQ ID NO: 79 - DNA, 35 bp, synthetic construct
  source 1..35: mol_type = other DNA
ggatccgcaacaactcagccatccacaccgtgcag

SEQ ID NO: 80 - DNA, 35 bp, synthetic construct; identical to SEQ ID NO: 79
  source 1..35: mol_type = other DNA

SEQ ID NO: 81 - DNA, 23 bp, synthetic construct
  source 1..23: mol_type = other DNA
ctgcacggtgtggatggctgagt

SEQ ID NO: 82 - DNA, 23 bp, synthetic construct
  source 1..23: mol_type = other DNA
ctgcacggtgtggatggctgagt

SEQ ID NO: 83 - DNA, 24 bp, synthetic construct
  source 1..24: mol_type = other DNA
tctgcggaccatccacaccgtgca

SEQ ID NO: 84 - DNA, 17 bp, synthetic construct
  source 1..17: mol_type = other DNA
tgtggatggtccgcaga

SEQ ID NO: 85 - DNA, 37 bp, synthetic construct
  source 1..37: mol_type = other DNA
gctcatcaacaagctccacggtggaggtacaaaggta

SEQ ID NO: 86 - DNA, 37 bp, synthetic construct; identical to SEQ ID NO: 85
  source 1..37: mol_type = other DNA

SEQ ID NO: 87 - DNA, 25 bp, synthetic construct
  source 1..25: mol_type = other DNA
tacctttgtacctccaccgtggagc

SEQ ID NO: 88 - DNA, 25 bp, synthetic construct
  source 1..25: mol_type = other DNA
tacctttgtacctccaccgtggagc

SEQ ID NO: 89 - DNA, 26 bp, synthetic construct
  source 1..26: mol_type = other DNA
aacggtaacggtggaggtacaaaggt

SEQ ID NO: 90 - DNA, 17 bp, synthetic construct
  source 1..17: mol_type = other DNA
cctccaccgttaccgtt

(19) United States
(12) Patent Application Publication
(10) Pub. No.: US 2023/0295706 A1
(43) Pub. Date: Sep. 21, 2023

(54) CASCADE OLIGONUCLEOTIDE DISPLACEMENT PROBES

(71) Applicants: Detect, Inc., Guilford, CT (US); Board of Regents, The University of Texas System, Austin, TX (US)

(72) Inventors: Spencer Glantz, West hartford, CT (US); Jonathan M. Rothberg, Miami Beach, FL (US); Xinghua Shi, Madison, CT (US); Benjamin Rosenbluth, New Haven, CT (US); Jaymin Patel, New Haven, CT (US); William A. Hansen, Clinton, CT (US); Jonathan Naccache, St-Mande (FR); Hope Kronman, New Haven, CT (US); Henry Kemble, Paris (FR); Caixia Lv, Guilford, CT (US); Andrew Ellington, Austin, TX (US); Sanchita Bhadra, Austin, TX (US)

(73) Assignees: Detect, Inc., Guiford, CT (US); Board of Regents, The University of Texas System, Austin, TX (US)

(21) Appl. No.: 18/153,577

(22) Filed: Jan. 12, 2023

Related U.S. Application Data
(60) Provisional application No. 63/298,867, filed on Jan. 12, 2022.

(51) Int. Cl. C12Q 1/6853; C12Q 1/6818

(52) U.S. Cl. CPC C12Q 1/6853; C12Q 1/6818

(57) ABSTRACT

Aspects of the disclosure relate to compositions and methods for amplifying and/or detecting one or more target nucleic acid sequences (e.g., a nucleic acid sequence of one or more pathogens) in a biological sample obtained from a subject. In some embodiments, the pathogens are viral, bacterial, fungal, parasitic, or protozoan pathogens, such as SARS-CoV-2 or an influenza virus. In some embodiments, the methods comprise isothermal amplification of a target nucleic acid and subsequent detection of the amplification products.

### RELATED APPLICATIONS

This application claims the benefit under 35 U.S.C. § 119(e) of the filing date of U.S. provisional Application Ser. No. 63/298,867, filed Jan. 12, 2022, entitled “CASCADE OLIGONUCLEOTIDE DISPLACEMENT PROBES,” the entire contents of which are incorporated herein by reference.

### REFERENCE TO AN ELECTRONIC SEQUENCE LISTING

The contents of the electronic sequence listing (H096670077US01-SEQ-KZM.xml; Size: 80,898 bytes; and Date of Creation: Jan. 9, 2023) is herein incorporated by reference in its entirety.

### FIELD

The disclosure generally relates to methods for detecting the presence of a target nucleic acid sequence.

### BACKGROUND

Most current methods for colorimetric lateral flow readout of Loop-mediated isothermal amplification (LAMP) amplicons rely on non-specific labeling methods such as incorporation of labeled primers or nucleotides. Not only do labeled primer-based detection methods suffer from the risk of false positives, they also are more likely to face false negative readout problems due to hook effects or steric occlusion of labels that get embedded within large LAMP amplicons.

### SUMMARY

Aspects of the disclosure relate to compositions and methods for amplifying and/or detecting target analytes (e.g., nucleic acids) in a sample. The disclosure is based, in part, on methods and compositions that produce nucleic acid reporters that result in high contrast, visible, signals (e.g., colored bands) when bound to certain immunoassay devices (e.g., lateral flow immunoassays). In some embodiments, compositions and methods described herein allow for direct application of amplification reactions including the nucleic acid reporters to immunoassay devices without any intervening fluid transfer or dilution steps.

Accordingly, in some aspects, the disclosure provides an amplification reaction probe set comprising a first hemi-duplex polynucleotide comprising a first polynucleotide strand hybridized to a second polynucleotide strand to form a duplex portion, each polynucleotide strand having a 5′ end and a 3′ end, the 5′ end of the first polynucleotide forming a target sequence-specific single stranded portion that extends past the 3′ end of the second polynucleotide strand, and the 3′ end of the second polynucleotide strand comprising a first label; and, a second hemi-duplex polynucleotide comprising a third polynucleotide strand hybridized to a fourth polynucleotide strand to form a duplex portion, each polynucleotide strand having a 5′ end and a 3′ end, the 3′ end of the third polynucleotide strand forming a single stranded portion having a region of complementarity to the duplex portion of the second polynucleotide strand of the first hemi-duplex polynucleotide and comprising a second label.

In some embodiments, a first polynucleotide strand and a second polynucleotide strand each independently range in length from about 10 nucleotides to about 50 nucleotides.

In some embodiments, a third polynucleotide strand and a fourth polynucleotide strand each independently range in length from about 10 nucleotides to about 50 nucleotides.

In some embodiments, a duplex region of a first hemi-duplex polynucleotide comprises a blunt end. In some embodiments, a duplex region of a second hemi-duplex polynucleotide comprises a blunt end.

In some embodiments, a duplex region of a first hemi-duplex polynucleotide comprises a GC clamp. In some embodiments, a duplex region of a second hemi-duplex polynucleotide comprises a GC clamp. In some embodiments, a GC clamp ranges in length from 1 nucleotide to about 10 nucleotides. In some embodiments, a GC clamp is 5 or 6 nucleotides in length.

In some embodiments, a duplex region of a first hemi-duplex polynucleotide comprises one or more branch migration domains. In some embodiments, a duplex region of a second hemi-duplex polynucleotide comprises one or more branch migration domains.

In some embodiments, a single stranded portion of a first hemi-duplex polynucleotide is at least three nucleotides longer than the GC clamp of the first hemi-duplex polynucleotide.

In some embodiments, a single stranded portion of a second hemi-duplex polynucleotide is the same length as the duplex portion of the second hemi-duplex polynucleotide.

In some embodiments, a target sequence-specific single stranded portion has a region of complementarity with a target polynucleotide. In some embodiments, a target polynucleotide is a Loop-mediated isothermal amplification (LAMP) amplicon. In some embodiments, a target sequence-specific single stranded portion has a region of complementarity with a target polynucleotide derived from a pathogen. In some embodiments, a target sequence-specific single stranded portion has a region of complementarity with a target polynucleotide derived from a human.

In some embodiments, a duplex portion of a second hemi-duplex polynucleotide does not have a region of complementarity with a target polynucleotide.

In some embodiments, a single stranded portion of a first hemi-duplex polynucleotide has a region of complementarity with a loop of a LAMP amplicon.

In some embodiments, a first label is selected from FAM, FITC, digoxigenin (DIG), dinitrophenyl (DNP), and biotin. In some embodiments, a second label is selected from FAM, FITC, digoxigenin (DIG), dinitrophenyl (DNP), and biotin.

In some aspects, the disclosure provides a dual-labeled molecule comprising a second polynucleotide strand of a first hemi-duplex polynucleotide hybridized to a third polynucleotide strand of a second hemi-duplex polynucleotide, as described by the disclosure.

In some embodiments, a first label of a dual-labeled molecule comprises FAM. In some embodiments, a second label of a dual-labeled molecule comprises biotin.

In some aspects, the disclosure provides an amplification mixture comprising an amplification reaction probe set as described by the disclosure; one or more buffering agents, one or more salts; a deoxynucleoside triphosphate (dNTP) mixture; a polymerase; and, optionally, one or more detergents and/or a reverse transcriptase.

In some embodiments, an amplification mixture further comprises one or more loop-mediated isothermal amplification (LAMP) primers. In some embodiments, an amplification mixture further comprises 2, 3, 4, 5, or 6 LAMP primers.

In some embodiments, an amplification mixture further comprises unlabeled amplification reaction probes corresponding to the amplification probe set as described herein, with the exception that the unlabeled probes do not comprise a first or second hapten.

In some embodiments, an amplification mixture comprises a ratio of labeled probes to unlabeled probes ranging from about 10:1 to about 1:10. In some embodiments, an amplification mixture comprises a ratio of labeled probes to unlabeled probes of about 1:6.

In some embodiments, an amplification mixture further comprises a target polynucleotide. In some embodiments, a target polynucleotide is derived from a pathogen. In some embodiments, a target polynucleotide is derived from a human.

In some aspects, the disclosure provides a method for producing dual-labeled detection products (e.g., dual-labeled molecules), the method comprising: performing an isothermal amplification reaction to amplify a target nucleic acid in the presence of a first hemi-duplex polynucleotide comprising a first polynucleotide strand hybridized to a second polynucleotide strand to form a duplex portion, each polynucleotide strand having a 5′ end and a 3′ end, the 5′ end of the first polynucleotide forming a target nucleic acid sequence-specific single stranded portion that extends past the 3′ end of the second polynucleotide strand, and the 3′ end of the second polynucleotide strand comprising a first label; a second hemi-duplex polynucleotide comprising a third polynucleotide strand hybridized to a fourth polynucleotide strand to form a duplex portion, each polynucleotide strand having a 5′ end and a 3′ end, the 3′ end of the third polynucleotide strand forming a single stranded portion having a region of complementarity to the duplex portion of the second polynucleotide strand of the first hemi-duplex polynucleotide and comprising a second label; one or more additional primers that bind to the target nucleic acid; a deoxynucleoside triphosphate (dNTP) mixture; a polymerase; and, optionally, a reverse transcriptase; and producing dual-labeled detection products by using an oligonucleotide strand displacement reaction to form a duplex molecule comprising the second polynucleotide strand of the first hemi-duplex molecule and the third polynucleotide strand of the second hemi-duplex molecule.

In some embodiments, the isothermal amplification reaction is loop-mediated isothermal amplification (LAMP). In some embodiments, the one more additional primers comprises 2, 3, 4, 5, or 6 additional primers. In some embodiments, the one or more additional primers (e.g., 2, 3, 4, 5, or 6 additional primers) are LAMP primers.

In some embodiments, a target nucleic acid is derived from a pathogen. In some embodiments, a target nucleic acid is derived from a human.

In some aspects, the disclosure provides a method for indirectly detecting isothermal amplification of a target nucleic acid in a reaction, the method comprising producing one or more dual-labeled detection products according to a method as described by the disclosure; and contacting a lateral flow assay (LFA) device with the one or more dual-labeled detection products to produce one or more detectable signals; and identifying the presence of the target nucleic acid in the isothermal amplification reaction based upon detecting the presence of the detectable signal produced by the one or more dual-labeled detection products.

In some embodiments, the target nucleic acid is derived from a pathogen. In some embodiments, the target nucleic acid is derived from a human.

In some aspects, the disclosure provides an isolated nucleic acid comprising the sequence set forth in any one of SEQ ID NOs: 1-90. In some embodiments, the disclosure provides a set of cOSD probes comprising 1, 2, 3, 4, 5, 6, 7, 8, or more of the nucleic acids set forth in any one of SEQ ID NOs: 1-90.

### BRIEF DESCRIPTION OF THE DRAWINGS

FIGS. 1A-1C show an exemplary diagram of OSD cascade probes (also referred to as cascade OSD probes or cOSD probes). FIG. 1A shows production of dual-labeled lateral flow assay (LFA) products using cOSD probes. FIG. 1B depicts probe sequence regions and labels.

FIG. 1C shows the expected states of the probes in the presence and absence of the target amplicon. The different sequence regions of the probes are designed to maximize the formation of the A-S:B-L complex in the presence of the target, and minimize it otherwise.

FIG. 2 shows LFA data for a functional OSD cascade. The left panel shows addition of short (5-6 bp) GC rich ‘clamp’ regions with high melting temperature at the duplex end of both OSD molecules in the cascade prevents strand exchange in the absence of on-target amplification. The right panel shows addition of dual-labeled products created by cOSDs to LFA strips.

FIG. 3 shows representative data indicating that a 1:6 ratio between the unlabeled and labeled cOSD probes prevents LFA bands from being produced in no template control (NTC) samples directly added to Milenia T2 and N6 LFA strips without being diluted.

FIG. 4 shows representative data for the effect of probe concentration on band intensity. Two different annealing ratios of labeled:unlabeled strands were tested, in addition to a titration of each set of probes. Target LAMP reaction was Human RNase P primer set with RNase P template, whereas unspecific LAMP was SARS-CoV-2 primer set with an irrelevant template (CV).

FIG. 5 shows representative data for evaluation of target loop primer concentration on cOSD probes sensitivity. An RNase P template titration (2-fold serial dilution) was used to compare sensitivity of cOSD probes against labelled primers. Without the target loop primer, sensitivity was impacted with a detection limit near 3 copies/4 whereas when the LB loop primer was added to 50% relative to the LF primer, no difference between labelled primers and cOSD probes was observed.

FIG. 6 shows representative data for evaluation of cOSD probes on 1× frozen nasal swabs. Four confirmed COVID-negative frozen swabs were pooled together, and split between labelled primers and cOSD probes in a head-to-head comparison. Reactions were carried in 300 μl volume, and incubated in a warmer. Detection of human control was 8/8 in both labelled primers and Human RNase P-LB-cOSD4 probe. Also, no false positive from SARS-CoV-2-LB-cOSD2 (0/8) nor SARS-CoV-2-LB-cOSD7 (0/3) was observed.

FIG. 7 shows a schematic of experimental design using contrived samples and LFA readout devices. An experiment using pooled nasal material from frozen swabs and heat inactivated COVID virus was carried out to simulate potential product performance. Virus was used at 5,000 copies/swab.

FIG. 8 shows representative data for evaluation of the impact of probe and primer concentrations on COVID (CV) detection sensitivity. Probes Human RNase P-LB-cOSD4 and SARS-CoV-2-LB-cOSD7 were used. The concentration of the SARS-CoV-2 primer and its LB-primer were varied against a titration of CV synthetic RNA template concentrations. The percentages of LB primers represent the values relative to the LF primer. Both rows with 2× SARS-CoV-2 primers also contained twice the amount of SARS-CoV-2 probes relative to the 1×SARS-CoV-2 conditions.

### DETAILED DESCRIPTION

The disclosure relates, in some aspects, to compositions and methods for amplifying and/or detecting target analytes (e.g., nucleic acids) in a sample. The disclosure is based, in part, on cascade oligonucleotide strand displacement probes (cOSDs) that improve the specificity, sensitivity, and signal processing capacity for readout of isothermally generated nucleic acid amplicons. In particular, cascade OSDs enable sequence-specific, colorimetric, lateral flow readout of loop-mediated isothermal amplification (LAMP), as opposed to previously described techniques that rely on non-specific detection using labeled primers.

In some embodiments, cOSDs described by the disclosure overcome ‘hook effects’ during lateral flow assays. In some embodiments, dual-labeled molecules produced by cOSDs can be directly analyzed (e.g., by LFA) without requiring additional steps of label dilution prior to being analyzed (e.g., prior to LFA readout). Compared to other methods, such as labeled primers, for colorimetric lateral flow LAMP readout, OSD cascades produce more intensely colored lateral flow test lines, thereby, reducing false negatives.

In some aspects, the disclosure describes compositions and methods for the direct application of dual-labeled molecules (e.g., dual-labeled molecules produced by reacting cOSDs with LAMP amplicons) to lateral flow strips without any intervening dilution or sample preparation steps.

Cascade Oligonucleotide Strand Displacement (cOSD) Probes

Aspects of the disclosure relate to the recognition that inclusion of certain oligonucleotide strand displacement (OSD) probes in isothermal amplification reactions (e.g., LAMP) allows for direct application of the resulting amplicon mixtures to immunoassay devices without any intervening fluid transfer or dilution steps. A “cascade oligonucleotide strand displacement probe” or “cOSD” generally refers to one or more modified polynucleotides (e.g., a set of modified polynucleotides) that participate in chemical reaction cascades resulting in transduction of signals from long DNA amplicons generated by isothermal amplification techniques (e.g., LAMP) into short dual-labeled molecules (e.g., dual-labeled DNA duplex molecules) that can then be captured by colorimetric readout, for example by a lateral flow assay (LFA).

A set of cascade OSDs generally comprises two or more hemi-duplex polynucleotides (e.g., 2, 3, 4, 5, or more hemi-duplex polynucleotides). In some embodiments, a cOSD probe set comprises (or consists of) two cOSD probes. Each of the two or more hemi-duplex polynucleotides of a cOSD probe set comprises a region of complementarity with the other probes of the set, such that when one strand of a first probe is displaced during an isothermal amplification reaction, the displaced probe strand mediates a second oligonucleotide strand displacement reaction with another probe of the set, thereby forming a dual-labeled duplex molecule comprising one strand from each of the two cOSD probes involved in the isothermal amplification and second strand displacement reactions. In some embodiments, a set of cOSD probes comprises 1, 2, 3, 4, 5, 6, 7, 8, or more isolated nucleic acids selected from the isolated nucleic acids set forth in Table 4.

Although the following descriptions of cOSD probes (e.g., first hemi-duplex polynucleotides and second hemi-duplex polynucleotides) are described as having certain structural features, such as a single stranded portion, toehold, duplex region, GC clamp, label, etc., on a “5′ end” or a “3′ end”, the skilled artisan recognizes that the disclosure also contemplates embodiments in which those cOSD probe structural features are arranged in alternative orientations. For example, a label described as being present at a 5′ end may also be present, in other embodiments, at a 3′ end. In another example, a single stranded portion described as being present at a 5′ end may, in other embodiments, be present at a 3′ end.

In some embodiments, the first hemi-duplex polynucleotide of a cOSD probe set comprises a first polynucleotide strand hybridized to a second polynucleotide strand to form a duplex portion. The length of each strand of a first hemi-duplex polynucleotide may vary. In some embodiments, each polynucleotide strand of the first hemi-duplex polynucleotide ranges from about 10 to about 50 nucleotides in length. In some embodiments, each strand of the hemi-duplex polynucleotide is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

The duplex portion is formed by hybridization between the two polynucleotide strands such that the first strand is in a 5′ to 3′ orientation, and the second strand is in a 3′ to 5′ orientation (e.g., the strands of the duplex portion of the hemi-duplex molecule are reverse complements of one another). In some embodiments, the duplex region of a first hemi-duplex polynucleotide ranges from about 5 nucleotides to about 40 nucleotides in length. In some embodiments, the duplex region is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a duplex region comprises a blunt end. The blunt end may be the 5′ end of the duplex or the 3′ end of the duplex (e.g., relative to the sense strand of the first hemi-duplex polynucleotide).

In some embodiments, the 5′ end of the first polynucleotide strand of a first hemi-duplex polynucleotide comprises a toehold region. A “toehold region” is a single stranded portion of a hemi-duplex polynucleotide that comprises the region of complementarity with a target nucleic acid. In some embodiments, a single stranded toehold region ranges from about 5 to about 45 nucleotides in length. In some embodiments, the toehold region is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length. In some embodiments, a toehold region is a target sequence-specific single stranded portion that extends past the 3′ end of the second polynucleotide strand of a first hemi-duplex polynucleotide. In some embodiments, a toehold region comprises a region of complementarity that is at least 50%, 60%, 70%, 80%, 90%, 95%, 99%, or 100% complementary to a target nucleic acid. In some embodiments, a toehold region comprises a region of complementarity with a target nucleic acid, where the region of complementarity comprises 1, 2, 3, 4, 5, or more mismatches between the toehold region and the target nucleic acid.

In some embodiments, a first strand or a second strand of a first hemi-duplex polynucleotide comprises one or more modifications (e.g., 2. 3, 4, or more modifications). A modification may comprise a blocking group (e.g., an extension blocking group, a label, a hapten, or any combination of the foregoing). Examples of blocking groups include but are not limited to a dehydroxylated 3′ nucleotide, 3′ dideoxycytosine (ddC), 3′ inverted deoxythymidine (dT), 3′ propyl (C3) spacer, 3′ amino modification, 3′ phosphoryl modification, or 3′ biotin modification. Examples of labels include but are not limited to FAM, FITC, digoxigenin (DIG), dinitrophenyl (DNP), and biotin. In some embodiments, first hemi-duplex polynucleotide comprises a modification on the 3′ end of the first polynucleotide strand of the first hemi-duplex polynucleotide. In some embodiments, the modification comprises an extension blocking group. In some embodiments, first hemi-duplex polynucleotide comprises a modification on the 5′ end of the second polynucleotide strand of the first hemi-duplex polynucleotide. In some embodiments, the modification comprises an extension blocking group. In some embodiments, a first hemi-duplex polynucleotide comprises a modification on the 3′ end of the second polynucleotide strand of the first hemi-duplex polynucleotide. In some embodiments, the modification comprises a label selected from FAM, FITC, digoxigenin (DIG), dinitrophenyl (DNP), and biotin. In some embodiments, the label is FAM. In some embodiments, any terminus (e.g., 3′ end or 5′ end) of a nucleic acid strand of a hemi-duplex polynucleotide that does not comprise a label comprises an extension blocking agent.

In some embodiments, a cOSD probe set comprises a second hemi-duplex polynucleotide. In some embodiments, a second hemi-duplex polynucleotide comprises a third polynucleotide strand hybridized to a fourth polynucleotide strand to form a duplex portion. The length of each strand of a second hemi-duplex polynucleotide may vary. In some embodiments, each polynucleotide strand of the second hemi-duplex polynucleotide ranges from about 10 to about 50 nucleotides in length. In some embodiments, each strand of the second hemi-duplex polynucleotide is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

The duplex portion of the second hemi-duplex polynucleotide is formed by hybridization between the two polynucleotide strands (e.g., the third and fourth polynucleotide strands) such that the third strand is in a 5′ to 3′ orientation, and the fourth strand is in a 3′ to 5′ orientation (e.g., the strands of the duplex portion of the second hemi-duplex molecule are reverse complements of one another). In some embodiments, the duplex region of a second hemi-duplex polynucleotide ranges from about 5 nucleotides to about 40 nucleotides in length. In some embodiments, the duplex region is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a duplex region comprises a blunt end. The blunt end may be the 5′ end of the duplex or the 3′ end of the duplex (e.g., relative to the sense strand of the first hemi-duplex polynucleotide).

In some embodiments, the 3′ end of the third polynucleotide strand of a second hemi-duplex polynucleotide comprises a single stranded portion having a region of complementarity to the duplex portion of a second polynucleotide strand (e.g., a duplex-forming portion of the second strand) of a first hemi-duplex polynucleotide. The length of the single stranded portion may vary. In some embodiments, a single stranded portion ranges from about 5 to about 45 nucleotides in length. In some embodiments, the single stranded portion is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length. In some embodiments, a single stranded portion extends past the 5′ end of the fourth polynucleotide strand of a second hemi-duplex polynucleotide. In some embodiments, a single stranded portion comprises a region of complementarity that is at least 50%, 60%, 70%, 80%, 90%, 95%, 99%, or 100% complementary to a portion (e.g., a duplex-forming portion) of a second polynucleotide strand of a first hemi-duplex polynucleotide. In some embodiments, a toehold region comprises a region of complementarity with a target nucleic acid, where the region of complementarity comprises 1, 2, 3, 4, 5, or more mismatches between the toehold region and the target nucleic acid.

In some embodiments, a third strand or a fourth strand of a second hemi-duplex polynucleotide comprises one or more modifications (e.g., 2. 3, 4, or more modifications). A modification may comprise a blocking group (e.g., an extension blocking group, a label, a hapten, or any combination of the foregoing). Examples of blocking groups include but are not limited to a dehydroxylated 3′ nucleotide, 3′ dideoxycytosine (ddC), 3′ inverted deoxythymidine (dT), 3′ propyl (C3) spacer, 3′ amino modification, 3′ phosphoryl modification, or 3′ biotin modification. Examples of labels include but are not limited to FAM, FITC, digoxigenin (DIG), dinitrophenyl (DNP), and biotin. In some embodiments, second hemi-duplex polynucleotide comprises a modification on the 5′ end of the third polynucleotide strand of the second hemi-duplex polynucleotide. In some embodiments, the modification comprises an extension blocking group. In some embodiments, second hemi-duplex polynucleotide comprises a modification on the 3′ end of the fourth polynucleotide strand of the second hemi-duplex polynucleotide. In some embodiments, the modification comprises an extension blocking group. In some embodiments, a second hemi-duplex polynucleotide comprises a modification on the 3′ end of the third polynucleotide strand of the second hemi-duplex polynucleotide. In some embodiments, the modification comprises a label selected from FAM, FITC, digoxigenin (DIG), dinitrophenyl (DNP), and biotin. In some embodiments, the label is biotin.

The disclosure is based, in part, on the recognition that inclusion of a GC clamp in cOSD probes improves the kinetics of the oligonucleotide strand displacement cascades mediated by the cOSD probes. A “GC” clamp generally refers to addition of one or more guanine (G) or cytosine (C) bases in the terminal five bases of a polynucleotide. In some embodiments, a duplex region (e.g., one or both strands of a first hemi-duplex polynucleotide or a second hemi-duplex polynucleotide) comprises a GC clamp. In some embodiments, a GC clamp ranges in length from 1 nucleotide to about 10 nucleotides (e.g., 1, 2, 3, 4, 5, 6, 7, 8, 9, or 10 nucleotides). In some embodiments, a GC clamp comprises or consists of 5 or 6 G and/or C nucleotides.

A first hemi-duplex polynucleotide and a second hemi-duplex polynucleotide may comprise one or more branch migration domains. A “branch migration domain” generally refers to a polynucleotide sequence that is capable of being exchanged (e.g., via recombination, hybridization, etc.) with a homologous sequence of another polynucleotide molecule. For example, in the context of a cOSD probe set, the duplex portion of the first hemi-duplex polynucleotide may comprise a branch migration domain that is homologous to a branch migration domain of the single stranded portion of the second hemi-duplex polynucleotide. In some embodiments, the branch migration domain of the first hemi-duplex polynucleotide is blocked or occluded (e.g., not available for hybridization) with respect to the single stranded branch migration domain of the second hemi-duplex domain when there is no target nucleic acid (e.g., LAMP amplicon) present. In some embodiments, presence of a target nucleic acid (e.g., a LAMP amplicon) results in ‘unblocking’ of the branch migration domain of the first hemi-duplex polynucleotide via an oligonucleotide strand displacement reaction involving the target nucleic acid and the first hemi-duplex polynucleotide. In some embodiments, a target-specific single stranded region of a first hemi-duplex polynucleotide is at least 3 (e.g., 3, 4, 5, 6, 7, 8, or more) nucleotides longer than the combined length of i) a branch migration domain of the first hemi-duplex polynucleotide and 2) a GC clamp of the first hemi-duplex polynucleotide.

### Nucleic Acids

The disclosure relates, in some aspects, to nucleic acids and nucleic acid sequences. A “nucleic acid” sequence refers to a DNA or RNA (or a sequence encoded by DNA or RNA). In some embodiments, a nucleic acid is isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which can be manipulated by recombinant DNA techniques well known in the art. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In some embodiments, a nucleic acid or isolated nucleic acid is referred to as a “polynucleotide” or “oligonucleotide”. The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-O-methyl-RNA, or combinations thereof.

The length of a polynucleotide (or each strand of a double stranded, hemi-duplex, or duplex molecule) may vary. In some embodiments, a polynucleotide ranges from about 2 to 10, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 2000, 2 to 5000, 2 to 10,000, 2 to 50,000, 2 to 500,000, or 2 to 1,000,000 nucleotides in length. In some embodiments, a polynucleotide is more than 1,000,000 nucleotides in length (e.g., longer than a megabase).

A nucleic acid (e.g., a polynucleotide) may be single stranded or double stranded. In some embodiments, a single stranded polynucleotide comprises a sequence of polynucleotides connected by a contiguous backbone. In some embodiments, a single stranded polynucleotide comprises a 5′ portion (end or terminus) and a 3′ portion (end or terminus). A single stranded polynucleotide may be a sense strand or an antisense strand.

In some embodiments, a nucleic acid (e.g., polynucleotide) is double stranded. A double stranded polynucleotide comprises a first (e.g., “sense”) polynucleotide strand that is hybridized to a second polynucleotide (“antisense”) strand via hydrogen bonding between the nucleobases of each strand along a region of complementarity between the two strands. Each strand of a double stranded polynucleotide comprises a 5′ portion and a 3′ portion.

As used herein, the term “region of complementarity” refers to a region of a nucleic acid (e.g., polynucleotide) that is substantially complementary (e.g., forms Watson-Crick base pairs) to another nucleic acid sequence, for example a target nucleic acid sequence, control nucleic acid sequence, or a corresponding antisense strand (e.g., in the case of a double stranded or duplex nucleic acid). Two nucleic acids, for example a sense strand and antisense strand of a double stranded polynucleotide, may comprise a region of complementarity that ranges from about 70% to about 100% complementarity. In some embodiments, two polynucleotides share a region of complementarity that is about 50%, 60%, 70%, 80%, 90%, 95%, 99%, or 100% complementary. In some embodiments, a region of complementarity between two nucleic acids comprises at least 1, 2, 3, 4, 5, 6, 7, 8, 9, 10, 15, 20, 25, 50, 100, 200, or 500 mismatched nucleotide bases. In some embodiments, a region of complementarity between two nucleic acids comprises between 1 and 5, 2 and 10, 5 and 15, 10 and 50, 30 and 100, or 75 and 500 mismatched nucleotide bases. Mismatched nucleotide bases within a region of complementarity may result in the resulting complex comprising one or more bulges or loops.

In some embodiments, a single stranded polynucleotide or a double stranded polynucleotide forms a duplex region. A “duplex” refers to a stable complex formed by fully or partially complementary polynucleotides that undergo Watson-Crick type base pairing along a region of complementarity. For example, a sense strand and its reverse-complement antisense strand form a duplex molecule when hybridized (e.g., annealed) together. In another example, a single stranded polynucleotide forms a duplex molecule when two portions of the polynucleotide self-hybridize to form a stem-loop (e.g., hairpin) structure.

A duplex molecule may comprise one or two blunt ends. A “blunt end” refers to a double stranded polynucleotide (e.g., a duplex molecule) where the one end of the first (sense) polynucleotide strand and one end of the second (e.g., antisense) strand terminate at the same nucleotide position such that no overhang is formed. A blunt end may be formed at either the 5′ end or the 3′ end (e.g., with respect to the sense strand) of a duplex molecule. In some embodiments, a duplex molecule comprises two blunt ends. In some embodiments, a duplex molecule comprises one blunt end and one overhang. An “overhang” refers to a single stranded region of a duplex molecule formed by asymmetric hybridization of the first (e.g., sense) and second (e.g., antisense) strands of a duplex. Asymmetric hybridization may result from a difference in the lengths of two polynucleotides forming the duplex (e.g., one polynucleotide is longer than another) or from the two polynucleotide strands sharing a region of complementarity that does not encompass the entire length of one (or both) of the polynucleotides. Thus, in some embodiments, a duplex molecule comprises two overhangs.

In some embodiments, a double stranded polynucleotide having an overhang is a hemi-duplex molecule. As used herein, “hemi-duplex” refers to a double stranded polynucleotide molecule in which one polynucleotide strand is longer than the other polynucleotide strand, such that when the strands are hybridized (or annealed) to one another, the resulting double stranded molecule comprises a single stranded portion and a double stranded portion. In some embodiments, the single stranded portion of a hemi-duplex molecule is referred to as a “toehold” portion of the hemi-duplex.

The length of an overhang may vary. In some embodiments, an overhang ranges from between 1 and 100 nucleotides in length. In some embodiments, an overhang ranges from between 1 and 5 nucleotides in length. In some embodiments, an overhang ranges from between 2 and 10, 5 and 15, 10 and 25, or 20 and 100 nucleotides in length. In some embodiments, an overhang is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a nucleic acid (e.g., polynucleotide) is a primer. As used herein, a “primer” refers to a polynucleotide that is capable of selectively binding (e.g., hybridizing or annealing) to a nucleic acid template and allows the synthesis of a sequence complementary to the corresponding polynucleotide template. A nucleic acid template may be a target nucleic acid or control nucleic acid. In some embodiments, a nucleic acid template comprises a region of complementarity with one or more primers. Generally, a primer ranges in size from about 10 to 100 nucleotides, and functions as a point of initiation for template-directed, polymerase mediated synthesis of a polynucleotide complementary to the template. In some embodiments, a primer is specific for a target nucleic acid. In some embodiments, a primer is specific for a control nucleic acid. A primer that is “specific” for a template binds (e.g., hybridizes) to that template with higher affinity than any other nucleic acid. In some embodiments, a primer that is “specific” for a template does not bind to any other nucleic acids present in a sample along with the template. A “pair of primers” or “primer pair” refers to two primers that bind to different regions (or portions) of the same template (e.g., the same target nucleic acid).

In some embodiments, a primer is an outer primer. An “outer primer” refers to a primer that binds at or near a 5′ end (e.g., at or near the 5′ terminal nucleotide) of a template sequence (e.g., a target nucleic acid, control nucleic acid, etc.), or at or near a 3′ end (e.g., at or near the 3′ terminal nucleotide) of a template sequence (e.g., a target nucleic acid, control nucleic acid, etc.) and is capable of displacing, or un-hybridizing, the two strands of a duplex upon hybridization of the primer to one of the duplex strands. In some embodiments, an outer primer binds to a terminal nucleotide, such as a 5′ terminal nucleotide or a 3′ terminal nucleotide of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 5′ end (terminal nucleotide) of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 3′ end (terminal nucleotide) of a template. In some embodiments, an outer primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a primer is an internal primer. An “internal primer” refers to a primer that binds internally to an outer primer (e.g., downstream or 3′ relative to a 5′ outer primer, or upstream or 5′ relative to a 3′ outer primer). In some embodiments, internal primers are referred to as “nested primers”. In some embodiments, an internal primer binds to nucleic acid sequence that is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of an outer primer. In some embodiments, an internal primer binds more than 50 nucleotides away from an outer primer, for example 60, 70, 80, 90, 100, 150, 200, or more nucleotides upstream or downstream of an outer primer. In some embodiments, an internal primer specifically hybridizes to a target nucleic acid. In some embodiments, an internal primer specifically hybridizes to a control nucleic acid. In some embodiments, an internal primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, an internal primer is a loop primer. A “loop primer” refers to a primer that binds to a single stranded duplex (e.g., loop) region of a template nucleic acid that is formed during Loop-mediated isothermal amplification (LAMP). In some embodiments, loop primers accelerate amplification (e.g., decrease the reaction time) during LAMP. In some embodiments, a loop primer binds to a region of a template (e.g., target nucleic acid) that is downstream of (e.g., 3′ relative to) an outer primer or another internal primer. In some embodiments, a loop primer binds to a region of a template (e.g., target nucleic acid) that is upstream of (e.g., 5′ relative to) an outer primer or another internal primer. In some embodiments, a loop primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

A nucleic acid may be unmodified or modified. A modified nucleotide may comprise one or more modified nucleic acid bases and/or a modified nucleic acid backbone. In some embodiments, a modified nucleic acid comprises one or more nucleotide analogs. The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza-nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, COOR, or, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

In some embodiments, a nucleic acid is modified by conjugation to one or more biological or chemical moieties. Examples of moieties used for modifying nucleic acids include fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof. In some embodiments, a nucleic acid (e.g., a primer) comprises one or more modifications. In some embodiments, a modified nucleic acid comprises a hapten. In some embodiments, the hapten is FITC. In some embodiments, the hapten is digoxigenin (DIG). In some embodiments, the hapten is biotin. In some embodiments, a modified nucleic acid comprises an extension blocking group. Examples of extension blocking groups include but are not limited to dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, a modified nucleic acid comprises both a hapten and an extension blocking group.

### Target Nucleic Acids

Methods and compositions described by the disclosure may be used, in some embodiments, to detect the presence or absence of any target nucleic acid sequence (e.g., from any pathogen of interest). In some embodiments, methods and compositions described herein comprise (or use) cascade OSD probes designed to interact with amplicons (e.g., LAMP amplicons) produced by isothermal amplification of a target nucleic acid sequence. Target nucleic acid sequences may be associated with a variety of diseases or disorders, as described below. In some embodiments, the compositions and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G mutation (i.e., a mutation of the 614th amino acid from aspartic acid (D) to glycine (G)) in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the devices, compositions, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2, SARS-CoV-2 D614G, an influenza type A virus, and/or an influenza type B virus).

In some embodiments, a composition (e.g., cOSD probe set or plurality of probe sets) or method is configured to detect at least 1, at least 2, at least 3, at least 4, at least 5, at least 6, at least 7, at least 8, at least 9, or at least 10 target nucleic acid sequences. In some embodiments, a composition comprises cOSD probe set(s) configured to detect 1 to 2 target nucleic acid sequences, 1 to 5 target nucleic acid sequences, 1 to 8 target nucleic acid sequences, 1 to 10 target nucleic acid sequences, 2 to 5 target nucleic acid sequences, 2 to 8 target nucleic acid sequences, 2 to 10 target nucleic acid sequences, 5 to 8 target nucleic acid sequences, 5 to 10 target nucleic acid sequences, or 8 to 10 target nucleic acid sequences. Each target nucleic acid sequence may independently be a nucleic acid of a pathogen (e.g., a viral, bacterial, fungal, protozoan, or parasitic pathogen) and/or a cancer cell. In some embodiments, a composition comprises a different cOSD probe set corresponding to each target nucleic acid sequence that is to be detected in a sample.

In some embodiments, the compositions and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or SARS-CoV-2 D614G. In certain embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabiá virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, the compositions and methods are configured to detect a target nucleic acid sequence of a bacterium (e.g., a bacterial pathogen). The bacterium may be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the compositions and methods are configured to detect a target nucleic acid sequence of a fungus (e.g., a fungal pathogen). Examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the compositions and methods are configured to detect a target nucleic acid sequence of one or more protozoa (e.g., a protozoan pathogen). Examples of protozoan pathogens include, but are not limited to, Entamoeba histolytica, Giardia lambda, Trichomonas vaginalis, Trypanosoma brucei, T. cruzi, Leishmania donovani, Balantidium coli, Toxoplasma gondii, Plasmodium spp., and Babesia microti.

In some embodiments, the compositions and methods are configured to detect a target nucleic acid sequence of a parasite (e.g., a parasitic pathogen). Examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, Schistosoma (liver fluke), Loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the compositions and methods are configured to detect a target nucleic acid sequence of a cancer cell. Cancer cells have unique mutations found in tumor cells and absent in normal cells. For example, the diagnostic devices, systems, and methods may be configured to detect a target nucleic acid sequence encoding a cancer neoantigen, a tumor-associated antigen (TAA), and/or a tumor-specific antigen (TSA). Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY—CO—I, RCAS1, SDCCAG16, TA-90 (Mac-2 binding protein\cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some embodiments, the compositions and methods are configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA1 and/or BRCA2 genes. In some instances, the diagnostic devices, systems, and methods are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the compositions and methods are configured to detect a target nucleic acid sequence associated with a genetic disorder. Non-limiting examples of genetic disorders include hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MY07A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the compositions and methods are configured to detect a target nucleic acid sequence of an animal pathogen. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

The compositions and methods described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic devices, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus. Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the diagnostic devices, systems, or methods described herein, one can determine whether the sample contains the one or more bacterial toxins. In some embodiments, the diagnostic devices, systems, or methods may be operated or conducted during the food production process to ensure food safety prior to consumption.

In some embodiments, the compositions and methods described herein may be used to test samples of soil, building materials (e.g., drywall, ceiling tiles, wall board, fabrics, wallpaper, and floor coverings), air filters, environmental swabs, or any other sample. In certain embodiments, the diagnostic devices, systems, and methods may be used to detect one or more toxins, as described above. In certain instances, the diagnostic devices, systems, and methods may be used to analyze ammonia- and methane-oxidizing bacteria, fungi or other biological elements of a soil sample. Such information can be useful, for example, in predicting agricultural yields and in guiding crop planting decisions.

### Control Nucleic Acid Sequences

In some embodiments, methods and compositions described herein comprise (or use) primers designed to amplify a human or animal nucleic acid that is not associated with a target nucleic acid from a pathogen, a cancer cell, or a contaminant. In some embodiments, methods and compositions described herein comprise (or use) cascade OSD probes designed to interact with amplicons (e.g., LAMP amplicons) produced from a human or animal nucleic acid that is not associated with a target nucleic acid from a pathogen, a cancer cell, or a contaminant. In some such embodiments, the human or animal nucleic acid may act as a control and is referred to as a “control nucleic acid”. For example, successful amplification and detection of a control nucleic acid may indicate that a sample was properly collected, and the diagnostic test was properly run (e.g., an amplification reaction was successful).

A control nucleic acid is typically a gene or portion of a gene that is widely expressed and/or expressed at a high level in a control organism (e.g., a human or other mammal). Such genes are typically referred to as “housekeeping genes”. Examples of housekeeping genes include but are not limited to GAPDH, B2M, ACTB, POLR2A, UBC, PPIA, HPRT1, GUSB, TBP, and H3F3A. In some embodiments, a control nucleic acid encodes a gene (or portion of a gene) selected from GAPDH, B2M, ACTB, POLR2A, UBC, PPIA, HPRT1, GUSB, TBP, H3F3A, POLR2A, RPLPO, L19, B2M, RPS17, ALAS1, CD74, RNase P, CK18, HMBS, IPO8, PGK1, YWHAZ, and STATH.

In some embodiments, a composition (e.g., a cOSD probe set or plurality of cOSD probe sets) or method is configured to detect at least 1, at least 2, at least 3, at least 4, at least 5, at least 6, at least 7, at least 8, at least 9, or at least 10 control nucleic acid sequences. In some embodiments, the cOSD probe set(s) is/are configured to detect 1 to 2 control nucleic acid sequences, 1 to 5 control nucleic acid sequences, 1 to 8 control nucleic acid sequences, 1 to 10 control nucleic acid sequences, 2 to 5 control nucleic acid sequences, 2 to 8 control nucleic acid sequences, 2 to 10 control nucleic acid sequences, 5 to 8 control nucleic acid sequences, 5 to 10 control nucleic acid sequences, or 8 to 10 control nucleic acid sequences. Each control nucleic acid sequence may independently be a nucleic acid of a human or another animal (e.g., mammal). In some embodiments, a composition comprises a different cOSD probe set corresponding to each control nucleic acid sequence that is to be detected in a sample.

### Methods of Amplifying Nucleic Acids

Aspects of the disclosure relate to methods and compositions for detecting target nucleic acids and/or control nucleic acids in a biological sample. In some embodiments, the methods comprise a step of performing an isothermal amplification reaction on a biological sample in order to amplify a target nucleic acid and/or a control nucleic acid.

The disclosure is based, in part, on amplification buffer solutions comprising one or more of the following components: an amplification reaction probe set (e.g., a cOSD probe set) as described by the disclosure; one or more buffering agents, one or more salts, and one or more detergents; a deoxynucleoside triphosphate (dNTP) mixture; a polymerase; and a reverse transcriptase. In some embodiments, the result of performing an amplification reaction using such a buffer is production of a mixture of amplification products (e.g., dual-labeled polynucleotide molecules) that can be directly added to a detection device, for example a lateral flow assay (LFA) device, and which results in visible, colored bands. This is surprising because it was previously thought that amplification mixtures needed to be diluted prior to addition to a detection device in order to produce brightly visible bands.

Thus, in some embodiments, following lysis, one or more target nucleic acids (e.g., a nucleic acid of a target pathogen) and/or one or more control nucleic acids may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, reverse transcription is performed by exposing lysate (or nucleic acids within a lysate) to one or more reverse transcription reagents. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid.

In some embodiments, a reverse transcriptase used for an isothermal amplification reaction (e.g., RT-LAMP) is selected from the group consisting of HIV-1 reverse transcriptase, Moloney murine leukemia virus (M-MLV) reverse transcriptase, and avian myeloblastosis virus (AMV) reverse transcriptase.

In some embodiments, DNA may be amplified according to any nucleic acid amplification method known in the art. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction.

In some embodiments, the isothermal amplification methods described below include a modified nucleotide, for example, deoxyuridine triphosphate (dUTP), during amplification. In such embodiments, a subsequent test may comprise a uracil-DNA glycosylase (UDG) treatment prior to the amplification step, followed by a heat inactivation step (e.g., 95° C. for 5 minutes) (Hsieh et al., Chem Comm, 2014, 50: 3747-3749). In some embodiments, the heat inactivation step may correspond to a thermal lysis step.

Without wishing to be bound by a particular theory, it is thought that the addition of dUTP during the amplification process will reduce or eliminate potential contamination between samples. In the absence of adding dUTP and UDG, amplicons may aerosolize and contaminate future tests, potentially result in false positive test results. The use of UDG (thermolabile Uracil DNA glycosylase) generally prevents carryover contamination by specifically degrading products have already been amplified (i.e., amplicons), leaving the unamplified (new) sample untouched and ready for amplification. Using this method, tests may be performed sequentially in the same tube and/or in the same area.

In some embodiments, an isothermal amplification method described herein comprises applying heat to a sample. In certain instances, an amplification method comprises applying an amplification heating protocol comprising heating the sample at one or more temperatures for one or more time periods using any heater described herein. In some embodiments, an amplification heating protocol comprises heating the sample at a first temperature for a first time period. In certain instances, the first temperature is at least 30° C., at least 32° C., at least 37° C., at least 50° C., at least 60° C., at least 65° C., at least 70° C., at least 80° C., or at least 90° C. In certain instances, the first temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes.

In some embodiments, an amplification heating protocol comprises heating the sample at a second temperature for a second time period. In certain instances, the second temperature is at least 30° C., at least 32° C., at least 37° C., at least 50° C., at least 60° C., at least 65° C., at least 70° C., at least 80° C., or at least 90° C. In certain instances, the second temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, an amplification heating protocol comprises heating the sample at a third temperature for a third time period. In certain instances, the third temperature is at least 30° C., at least 32° C., at least 37° C., at least 50° C., at least 60° C., at least 65° C., at least 70° C., at least 80° C., or at least 90° C. In certain instances, the third temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the third time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the third time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, a lysis heating protocol may comprise heating a sample at one or more additional temperatures for one or more additional time periods.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence. In some embodiments, LAMP is combined with a reverse transcription reaction, and referred to as RT-LAMP.

In some embodiments, the LAMP reagents comprise four or more primers (e.g., 4 or more primers in addition to the cOSD probes described in the sections above). In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be present in the sample.

In some embodiments, the LAMP reagents comprise a FIP and a BIP for one or more target nucleic acids. In some embodiments, the concentrations of FIP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FIP and BIP are each in a range from 0.5 μM to 1 μM, 0.5 μM to 1.5 μM, 0.5 μM to 2.0 μM, 1 μM to 1.5 μM, 1 μM to 2 μM, or 1.5 μM to 2 μM.

In some embodiments, the LAMP reagents comprise an F3 primer and a B3 primer for one or more target nucleic acids. In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acids. In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μm.

In some embodiments, the LAMP reagents comprise LAMP primers designed to amplify a human or animal nucleic acid that is not associated with a pathogen, a cancer cell, or a contaminant (e.g., a control nucleic acid). In some such embodiments, the human or animal nucleic acid may act as a control. For example, successful amplification and detection of the control nucleic acid may indicate that a sample was properly collected, and the diagnostic test was properly run.

In some embodiments, the control nucleic acid is a nucleic acid sequence encoding human RNase P.

The disclosure is based, in part, on LAMP amplification buffers comprising cOSD probes. In some embodiments, a LAMP amplification buffer comprises one or more target nucleic acid-specific cOSD probes. In some embodiments, a LAMP amplification buffer comprises one or more control nucleic acid-specific cOSD probes. In some embodiments, the first hemi-duplex polynucleotide of a cOSD probe binds (e.g., hybridizes) to the same portion of a target nucleic acid as an outer primer, inner primer, or loop primer. In some embodiments, the first hemi-duplex polynucleotide of a cOSD probe binds (e.g., hybridizes) to the same portion of a control nucleic acid as an outer primer, inner primer, or loop primer.

The disclosure is based, in part, on the recognition that inclusion of certain polynucleotides (e.g., polynucleotides corresponding to cOSD probes but lacking labels, also referred to as “unlabeled probes”) improves the signal-to-noise of LFA-based detection of dual-labeled polynucleotides produced by cOSD probes. In some embodiments, the ratio of concentration of labeled cOSD probes to unlabeled probes ranges from about 1:10 to about 10:1. In some embodiments, the ratio of concentration of unlabeled cOSD probes to labeled probes is about 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.5:1, 4.0:1, 4.5:1, 5.0:1, 5.5:1, 6:1, 6.5:1, 7.0:1, 7.5:1, 8.0:1, 8.5:1, 9.0:1, 9.5:1, or 10:1. In some embodiments, the ratio of concentration of labeled cOSD probes to unlabeled primers (e.g., LAMP primers) ranges from about 1:10 to about 10:1. In some embodiments, the ratio of concentration of labeled cOSD probes to unlabeled primers (e.g., LAMP primers) is about 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.5:1, 4.0:1, 4.5:1, 5.0:1, 5.5:1, 6:1, 6.5:1, 7.0:1, 7.5:1, 8.0:1, 8.5:1, 9.0:1, 9.5:1, or 10:1.

The ratio of two cOSD probe strands (e.g., a ratio of a first strand:second strand, first strand:third strand, first strand:fourth strand, second strand:third strand, second strand:fourth strand, third strand:fourth strand, etc.) in an amplification mixture may vary. In some embodiments, the ratio of concentration of one strand of a cOSD probe to another strand of a cOSD probe ranges from about 1:10 to about 10:1. In some embodiments, the ratio of one strand of a cOSD probe to another strand of a cOSD probe is about 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.5:1, 4.0:1, 4.5:1, 5.0:1, 5.5:1, 6:1, 6.5:1, 7.0:1, 7.5:1, 8.0:1, 8.5:1, 9.0:1, 9.5:1, or 10:1.

The amount or concentration of cOSD probes in an amplification mixture may vary. In some embodiments, the concentration of a first hemi-duplex polynucleotide in an amplification mixture is about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, or 25 nM. In some embodiments, the concentration of a second hemi-duplex polynucleotide in an amplification mixture is about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, or 25 nM.

In some embodiments, a buffer solution further comprises one or more sets (e.g., pairs) of outer primers. In some embodiments, at least one set (e.g. pair) of outer primers specifically binds to a target nucleic acid. In some embodiments, at least one set of outer primers specifically binds to a control nucleic acid.

In some embodiments, a buffer solution further comprises one or more additional sets (e.g., pairs) of internal primers. In some embodiments, at least one set (e.g., pair) of internal primers specifically binds to a target nucleic acid. In some embodiments, at least one set (e.g., pair) of internal primers specifically binds to a control nucleic acid.

In some embodiments, an amplification buffer comprises a DNA polymerase with high strand displacement activity. Non-limiting examples of suitable DNA polymerases include a DNA polymerase long fragment (LF) of a thermophilic-bacteria, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO₄ is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5M, 1.0 M to 1.2 M, or 1.0 M to 1.5M.

Aspects of the disclosure relate to methods of producing secondary reaction products during LAMP reactions that are useful surrogates for detection of LAMP amplicons during lateral flow assays (LFA). The disclosure is based, in part, on the use of cascade oligonucleotide strand displacement (cOSD) probes which allow for the direct application of reacted isothermal amplification (e.g., LAMP) mixtures to lateral flow strips without any intervening dilution or sample preparation steps. In some embodiments, the cOSD probes mediate secondary nucleic acid reactions (e.g., oligonucleotide strand displacement reactions) that result in the formation of controllable amounts of secondary products of desired size, structure and chemical labelling position/stoichiometry, optimized for performance as ideal LFA analytes according to these properties.

Accordingly, in some aspects, the disclosure provides a method for producing dual-labeled detection products, the method comprising: performing an isothermal amplification reaction to amplify a target nucleic acid in the presence of a first hemi-duplex polynucleotide comprising a first polynucleotide strand hybridized to a second polynucleotide strand to form a duplex portion, each polynucleotide strand having a 5′ end and a 3′ end, the 5′ end of the first polynucleotide forming a target nucleic acid sequence-specific single stranded portion that extends past the 3′ end of the second polynucleotide strand, and the 3′ end of the second polynucleotide strand comprising a first label; a second hemi-duplex polynucleotide comprising a third polynucleotide strand hybridized to a fourth polynucleotide strand to form a duplex portion, each polynucleotide strand having a 5′ end and a 3′ end, the 3′ end of the third polynucleotide strand forming a single stranded portion having a region of complementarity to the duplex portion of the second polynucleotide strand of the first hemi-duplex polynucleotide and comprising a second label; one or more additional primers that bind to the target nucleic acid; a deoxynucleoside triphosphate (dNTP) mixture; a polymerase; and a reverse transcriptase; and producing dual-labeled detection products by using an oligonucleotide strand displacement reaction to form a duplex molecule comprising the second polynucleotide strand of the first hemi-duplex molecule and the third polynucleotide strand of the second hemi-duplex molecule.

A “dual-labeled LFA detection product” or “dual-labeled molecule”, as used herein, refers to a double stranded oligonucleotide (e.g., a duplex molecule) comprising a second strand of a first hemi-duplex oligonucleotide of a cOSD probe pair hybridized to a third strand of a second hemi-duplex polynucleotide of a cOSD probe pair that is detectable by a lateral flow assay (LFA). In some embodiments, each label of a dual-labeled LFA detection product is selected from biotin, FAM, and DIG. The length of each strand of a dual-labeled detection product may vary. In some embodiments, each polynucleotide strand of the dual-labeled detection product ranges from about 10 to about 50 nucleotides in length. In some embodiments, each strand of the dual-labeled detection product is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, each end of the dual-labeled detection product comprises an overhang of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

### Detection

In some embodiments, dual-labeled detection products (e.g., representing amplification of a target or control nucleic acid) may be detected using any suitable methods. In some embodiments, one or more dual-labeled detection products (e.g., dual-labeled detection products representing one or more target nucleic acid sequences) are detected using a lateral flow assay strip. In some embodiments, one or more dual-labeled detection products (e.g., dual-labeled detection products representing one or more target nucleic acid sequences) are detected using a colorimetric assay.

Aspects of the disclosure relate to methods that result in detection of one or more signals on a lateral flow assay that are brighter (e.g., more intense, more easily visible to an unaided eye, etc.) than lateral flow assay signals produced using previous methods. In some embodiments, signals (e.g., colored bands) produced using isothermal amplification methods described herein are between 10% and 100% (10, 20, 30, 40, 50, 60, 70 80, 90, 95, 99, or 100%) brighter than lateral flow signals produced using previously available isothermal amplification methods.

In some embodiments, one or more target nucleic acid sequences are detected using a lateral flow assay strip. In some embodiments, a fluidic sample (e.g., fluidic contents of a reaction tube, a reagent reservoir, and/or a blister pack chamber) is transported through the lateral flow assay strip via capillary action. In some embodiments, the fluidic sample may comprise labeled amplicons.

In some embodiments, the fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as dual-labeled detection product-containing fluidic sample flows through the second sub-region (e.g., a particle conjugate pad), a labeled nanoparticle binds to a label of a dual-labeled detection product, thereby forming a particle-dual-labeled detection product conjugate.

In some embodiments, the fluidic sample (e.g., comprising a particle-dual-labeled detection product conjugate) subsequently flows through a third sub-region (e.g., a test pad) comprising one or more test lines. In some embodiments, a first test line comprises a capture reagent (e.g., an immobilized antibody) configured to detect a label present on the dual-labeled detection product. In some embodiments, a particle-dual-labeled detection product conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different dual-labeled detection product corresponding to a different target nucleic acid. In some instances, two or more test lines of the lateral flow assay strip are configured to detect the same dual-labeled detection product corresponding to the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a dual-labeled detection product representing amplification of a control nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the dual-labeled detection products were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip. Any excess fluid may flow through the fourth sub-region.

In some embodiments, the disclosure relates to rapid, self-administrable tests for detecting the presence of one or more target nucleic acids derived from one or more pathogens. A “self-administrable” test refers to a test in which all testing steps are performed by the subject of the test. In some embodiments, a self-administrable test is performed by a subject at a location that is not a medical facility (e.g., a hospital, physicians' office, nurse's office, etc.). In some embodiments, a self-administrable test is performed at the subject's home. In some embodiments, a “rapid test” refers to a test in which all testing steps (e.g., sample collection, lysis, isothermal amplification, detection, etc.) may be completed in less than 3 hours, less than 2 hours, or less than 1 hour. In some embodiments, a rapid test is completed (e.g., one of more nucleic acids are detected) in less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 minutes.

As an illustrative example, a fluidic sample comprising an dual-labeled detection product labeled with biotin and FITC may be introduced into a lateral flow assay strip (e.g., through a sample pad of a lateral flow assay strip). In some embodiments, as the dual-labeled detection product is transported through the lateral flow assay strip (e.g., through a particle conjugate pad of the lateral flow assay strip), a gold nanoparticle labeled with streptavidin may bind to the biotin label of the dual-labeled detection product. In some cases, the lateral flow assay strip (e.g., a test pad of the lateral flow assay strip) may comprise a first test line comprising an anti-FITC antibody. In some embodiments, the gold nanoparticle-dual-labeled detection product conjugate may be captured by the anti-FITC antibody, and an opaque band may develop as additional gold nanoparticle-dual-labeled detection product conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to a dual-labeled detection product) transported through the lateral flow assay strip may bind to the biotin of the first lateral flow control line, demonstrating that liquid was successfully transported to the first lateral flow control line. In some embodiments, one or more target nucleic acid sequences are detected using a colorimetric assay. In certain embodiments, for example, a fluidic sample is exposed to a reagent that undergoes a color change when bound to a dual-labeled detection product (e.g., a dual-labeled detection product representing amplification of viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the dual-labeled detection product representing the target nucleic acid is present, and the sample is positive. If the solution turns a different color (e.g., green), the dual-labeled detection product representing the target nucleic acid is not present, and the sample is negative. In some embodiments, the colorimetric assay may be a colorimetric LAMP assay; that is, the dual-labeled detection products may react in the presence or absence of a target nucleic acid sequence (e.g., from SARS-CoV-2) to turn one of two colors.

### Biological Samples

Aspects of the disclosure relate to methods for detecting one or more target nucleotides in a biological sample. In some embodiments, biological sample is obtained from a subject (e.g., a human subject, an animal subject). Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, a biological sample comprises blood, saliva, mucous, urine, feces, cerebrospinal fluid (CSF), or tissue.

In some embodiments, the biological sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds. In some embodiments, the biological sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020)—an amount that is detectable by any one of the methods described herein. In some embodiments, methods described herein are capable of detecting a concentration of a target nucleic acid (e.g., SARS-Cov-2 RNA) in a biological sample that is less than 5 fM.

In some embodiments, a biological sample (e.g., saliva sample) is deposited directly into a reaction tube. In some embodiments, the concentration of a target nucleic acid molecule (e.g., SARS-CoV-2 RNA) in the biological sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the biological sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of a target nucleic acid molecule (e.g., SARS-CoV-2 RNA) in the biological sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The biological sample, in some embodiments, is collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

A subject may be any mammal, for example a human, non-human primate (e.g., monkey, chimpanzee, ape, etc.), dog, cat, pig, horse, hamster, guinea pig, rat, mouse, etc. In some embodiments, a subject is a human. In some embodiments, a subject is an adult human (e.g., a human older than 16 years of age, 18 years of age, etc.). In some embodiments, a subject is a child (e.g., a pediatric subject), for example a subject that is less than 18 years of age, 16 years of age, etc. In some embodiments, a subject is an infant, for example a subject less than one year of age.

### EXAMPLES

### Example 1

This example describes rationally engineered oligonucleotide strand displacement (OSD) reaction cascades for signal transduction. Cascade OSDs (cOSDs) were designed for transducing long DNA amplicons generated by loop-mediated isothermal amplification (LAMP) into short dual labeled DNA duplexes that can then be captured by colorimetric readout. One of the principal uses of OSD cascades is as signal processors in enzymatic molecular diagnostics. For instance, OSD cascades can be used for signal transduction in LAMP reactions as described in this disclosure. OSD cascades may also be used to design logical signal processing pathways for information rich diagnostic tests, such as computation of amplicon co-presence, distinction of target variants, and higher order multiplex signal deconvolution via multicolored labels or distinctive spatial immobilization. The development of working OSD cascades simplifies the adaptation of designs to new analytes since initiating signaling via cOSDs is target-independent.

The minimum OSD cascade for colorimetric detection comprises (or consists of) two short LAMP amplicon-specific hemi-duplex DNA modules (termed OSD-A and OSD-B). OSD-A hemi-duplex is composed of a ‘long’ strand (“A-L”) bearing LAMP amplicon-specific toehold (domain 1) and branch migration (domains 2 and 3) domains bound to a complementary short strand (“A-S”) labeled with fluorescein (FAM). This short FAM strand also contains an OSD-B-specific toehold (domain 4*) that remains occluded in the absence of LAMP amplicons by base pairing to a complementary domain 4 in the long strand. OSD-B is also composed of a short strand (“B-S”) base paired to a long strand (“B-L”) that is labeled with biotin and bears a toehold complementary to OSD-A FAM strand (domain 4). Optimized toehold lengths, clamp domains (domains 2/2*, 5/5*, and 6/6*), and ratios of the four strands ensure negligible interaction of OSD-A and OSD-B in the absence of specific LAMP amplicons. When the target LAMP amplicon is present, OSD-A long strand undergoes toehold-mediated strand displacement hybridization to the LAMP loop releasing the short FAM strand and exposing its OSD-B toehold (domain 4*) in the process. The released FAM strand can now undergo toehold-mediated strand displacement hybridization with the biotinylated OSD-B long strand. The resulting FAM-biotin dual labeled complex can be colorimetrically visualized by sandwiching between a biotin ligand and a gold-labeled anti-fluorescein antibody at test lines of lateral flow dipsticks.

These OSD cascade design principles were used to rationally engineer four OSD cascades each specific to a distinct LAMP target—human fecal indicator HF183, E. coli, Drosophila C virus, and SARS-CoV-2. All four OSD cascades functioned as programmed in one-pot LAMP assays comprising all amplification and OSD cascade ingredients. Following LAMP amplification at 60° C.-65° C., when the reaction mixes were directly applied to lateral flow dipsticks, vivid color developed at test lines for reactions seeded with specific templates while reactions lacking templates or containing mutated templates remained negative. Compared to colorimetric detection by dual labeling of LAMP amplicons using fluorescein-OSD probes and biotinylated primers or nucleotides, signal transduction via OSD cascades required considerably lower concentration of labeled strands and yielded more intensely colored test lines. These results indicate that OSD cascades are not only adept at signal transduction but, by requiring very small amounts of labeled probes for readout, they also offer an effective approach to overcoming the lateral flow hook effect where excess labels, such as biotinylated primers, saturate the lateral flow reagents and falsely diminish signal intensity.

Four unique design features in the two component OSD cascade ensure specificity and sensitivity of this signal transduction reaction. First, in the OSD-A long strand, the target-specific toehold is three nucleotides longer than the combined length of the OSD-B-specific toehold (domain 4) and its adjoining clamp (domain 5). This design feature skews the first toehold-mediated strand displacement reaction towards target binding because hybridization of the OSD-A long strand to its specific LAMP amplicon will yield more base pairs and hence greater thermodynamic stability compared to the OSD-A hemi-duplex. This engineered binding preference improves signal strength.

Second, the OSD-C2 hemi-duplex is designed like a ““seesaw” gate”, in which the biotinylated long strand bears equal length toeholds for binding either the OSD-A FAM strand or the OSD-B short strand (domains 4 and 6, respectively). As a result, binding of the OSD-B biotinylated long strand to OSD-A FAM-strand or OSD-B short strand would be thermodynamically similar. This engineered equality in binding preference reduces cascade leakage by forcing OSD-A FAM strand to compete with the OSD-B short strand for binding the OSD-B biotinylated strand. Furthermore, the degree of this competition, and hence signal/noise, can be dialed by adjusting the ratios of the four OSD cascade strands in the system as well as by adding a LAMP loop primer whose binding region overlaps with that of the OSD cascade.

Third, the fact that the OSD-B toehold can be used between multiple different cascade designs may simplify the adaptation of these nucleic acid circuits to new targets. Different and orthogonal OSD-B (domain 4/4*) toehold pairs can also potentially be developed in advance in order to reduce crosstalk between multiplexed amplification reactions and circuits. This would simplify multiplexing as new targets or amplicons are included in a given reaction.

Fourth, the six nucleotide-long toehold (domain 4/4*) designed for mediating the strand displacement reaction between OSD-A FAM strand and OSD-B biotin strand was engineered using a random sequence without complementarity to the LAMP loop. This design feature thermodynamically favors formation of the dual label complex between the released OSD-A FAM strand and the OSD-B biotin strand as opposed to sequestration of these strands by hybridization of their domains 2*, 3* or 3 to LAMP amplicons. By deterring loss of labeled entities in unproductive hybridizations, this engineered thermodynamic bias maximizes signal from minimal amounts of labeled strands.

OSD cascades solve the problems of low sensitivity and specificity in lateral flow colorimetric readout of nucleic acids. Since small amounts of labeled OSD cascades are sufficient to produce a strong signal with minimal leakage, OSD cascades avoid lateral flow hook effects of excess labels, as seen in readout methods using labeled primers, and thereby increase readout sensitivity.

OSD cascades also improve readout system robustness by transducing LAMP amplicons into a small dual labeled lateral flow analyte. Unlike large LAMP amplicons whose movement on lateral flow devices is often slow or entirely constrained (depending on the dipstick construction), the small dual labeled OSD cascade analytes can move rapidly though most lateral flow systems. As a result, OSD cascades would reduce dependence on specially manufactured lateral flow devices and allow adoption of off-the shelf dipsticks from varied sources without significantly influencing readout.

OSD cascades also improve user experience and reduce complexity of test automation platforms. Since OSD cascades are designed to bypass hook effects, they allow direct lateral flow analysis of LAMP reactions unlike labeled primer-based colorimetric readout systems that require an additional step of dilution prior to lateral flow analysis in order to overcome hook effects. Removal of the dilution step streamlines test automation and eliminates a potential source of error. One less step in the test protocol also improves user experience.

OSD cascades provide a facile approach to overcoming label-dependent limits on multiplexing faced with current labeled primer-based methods for colorimetric readout. Labeled primer dependent readout methods are multiplexed mostly for up to two analytes. Parallel detection of additional analytes in this system would require the onerous task of generation and case-by-case optimization of additional orthogonal label:antibody pairs. In contrast, with the cascade OSD system it becomes possible to rationally build much larger multiplex systems based on universal design principles and only a single label:antibody pair. In one iteration, unlabeled OSD-B modules immobilized in a spatially barcoded grid can be used to capture amplicon-specific OSD-A strands that are all read visually using the same label:antibody conjugate.

OSD cascades also provide a programmable tool for higher order signal processing in multiplex reactions, for instance, logical computation of analyte variation, co-presence, or redundancy. The use of a common toehold in OSD-B may also allow improvements in multiplexing of LAMP-OSD reactions.

Currently, most methods for lateral flow LAMP readout rely on incorporation of labeled primers into the large LAMP amplicons, which are then captured on test lines of lateral flow devices. This method has several disadvantages including false positives due to non-specific amplification, false negatives due to hook effects, low signal due to label occlusion and constrained movement on lateral flow devices, and need for additional steps of sample dilution prior to lateral flow analysis. OSD cascades resolve all these problems of low sensitivity and specificity and also make it possible to directly analyze LAMP reactions by lateral flow without requiring sample dilution. OSD cascades can achieve this because of several design advantages over the current methods—first, by not being a part of the amplification process, OSD cascades effectively suppress signal from spurious amplicons; second, OSD cascades generate signal in a sequence-specific manner; third, by transducing the large LAMP amplicons into a small dual labeled lateral flow analyte, OSD cascades make the readout more sensitive and robust across different dipstick platforms; fourth, OSD cascades require very small amounts of labeled entities, thereby reducing both per reaction cost and hook effects that damage assay sensitivity; fifth, OSD cascades pave the way for facile multiplexing of multiple analytes with only a single label:antibody pair; sixth, OSD cascades open up the possibility of readily programmable higher order signal processing in multiplex reactions, for instance logical computation of analyte variation, co-presence, or redundancy; and seventh, LAMP multiplexing has proven to be challenging, and multiplex designs may be streamlined via OSD cascades.

### Example 2

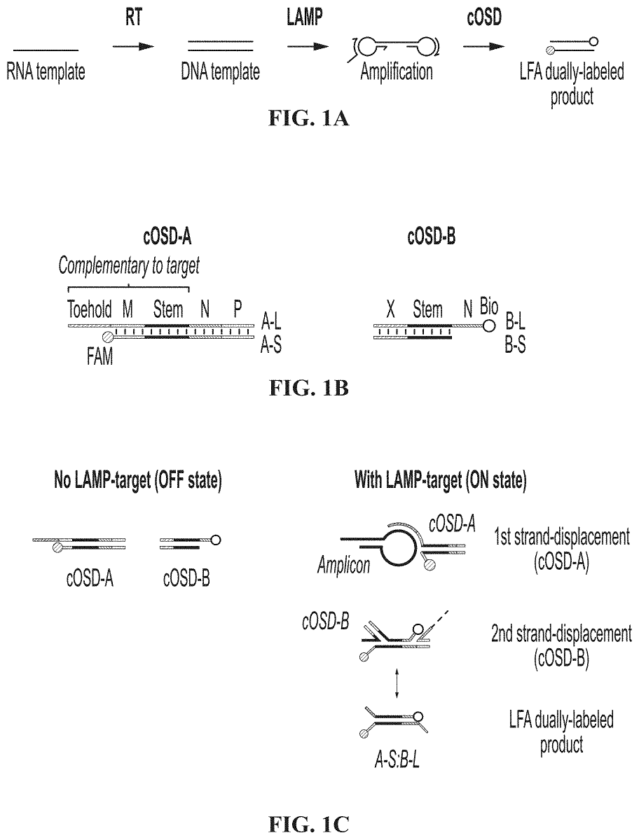

FIGS. 1A-1C show schematics depicting cOSDs. The OSD cascade (cOSD) strategy generally involves two steps: the first probe, cOSD-A, is a partially double-stranded DNA that in the presence of a free single-stranded region in the target amplicon (e.g. one of the loops of a LAMP amplicon), initiates a strand exchange reaction. One of those strands (the short strand, “A-S”) is labelled with a hapten, for example FAM or DIG or biotin. The second probe, cOSD-B, is also a partially double-stranded dsDNA probe, but features a distinct second label in one of the strands (the long strand, “B-L”). The cOSD-B probe serves as the target of the short strand of the cOSD-A (“A-S”) probe carrying the first label. After the first strand exchange is initiated in the presence of the target amplicon, a second strand-exchange follows leading to the formation of a dually labelled molecule suitable for LFA detection, namely the complex cOSD-A-S:cOSD-B-L.

There are some key advantages of the cOSD probes strategy relative to other LAMP probe techniques. First, cOSD probes are highly sequence specific, and have a high signal-to-noise ratio. Second, due to the nature of the cOSD probes, it is possible to add fluorescent labels and quenchers that report on the strand exchanges in real-time.

This example describes cOSD probe candidate design, and functional detection of SARS-CoV-2 and human RNaseP templates, both in single and multiplex reactions. Representative LAMP primer sets for SARS-CoV-2 and human RnaseP are shown in Tables 3 and 4. Furthermore, it was observed that detection of cascade OSDs in the presence of nasal material appears not to generate false positives. It was also observed that cOSDs are functional for multiplex detection of amplicons without the need of post-amplification dilution.

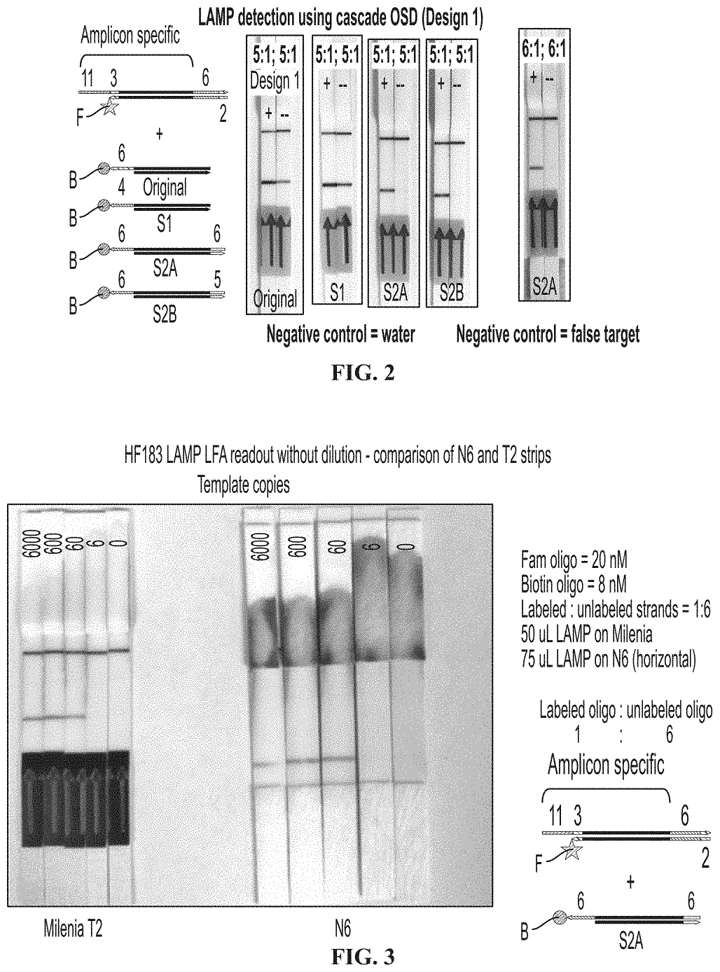

A functional OSD cascade that reduces off-target signaling was produced (FIG. 2) by the addition of short (5-6 bp) GC rich ‘clamp’ regions with high melting temperature at the duplex end of both OSD molecules in the cascade. Addition of the clamp regions greatly enhanced the overall specificity of the reaction. It was also observed that a 1:6 ratio between unlabeled and labeled strands worked well to prevent LFA bands without template (no template control, “NTC”).

A proof of concept reaction where the reacted RT-LAMP mix was applied directly to LFA strips with no intervening steps is shown in FIG. 3. Briefly, 20 nM FAM oligo (e.g., cOSD-A) and 8 nm biotin labeled oligo (cOSD-B) were used in a LAMP reaction. The ratio of labeled to unlabeled oligos was 1:6. Reaction mixtures containing LAMP products and dual-labeled molecules (e.g., resulting from cOSD strand exchange) were added directly (with no dilution) to either Milenia or N6 lateral flow assay media.

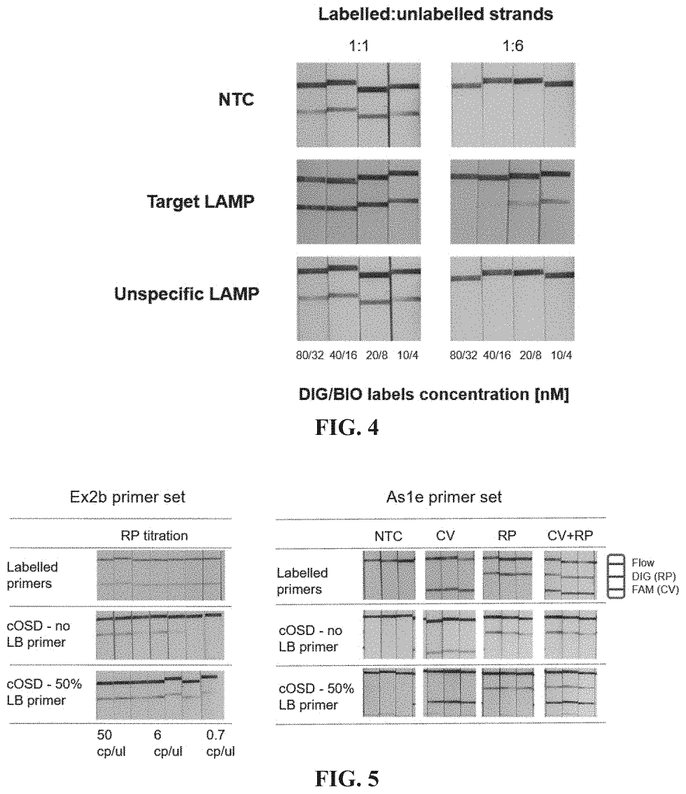

The concentration at which cOSD probes should be included in an isothermal amplification reaction was also investigated. Two candidates for two different primer sets (“SARS-CoV-2” and “Human RNase P”) were evaluated. Data for one candidate, Human RNase P-LB-cOSD4, are presented in FIG. 4, and illustrate how the ratio of labeled to unlabeled strands is an important parameter. A 1:1 ratio resulted in non-specific LFA bands at all concentrations tested. When using a 1:6 ratio of labeled to unlabeled strands, it was observed that band intensity increased with lower probe concentrations. This pattern was most likely not due to the saturation of the LFA strip, as similar concentrations on a 1:1 annealing ratio resulted in very bright LFA bands. Rather, at high probe concentrations, the toehold from the cOSD-A probe may interfere with the LAMP reaction, resulting in less bright bands due to a lack of LAMP product to initiate the cascade.

Six probe candidates for SARS-CoV-2 and four Human RNase P probes candidates were also tested on three conditions: no template (NTC), adding the target template of the target primer set, and adding the template from the other primer set (a non-specific LAMP control). In order to facilitate the interpretation of the results and prevent the chance of template contaminations, only the needed primer sets were added for testing each condition. Representative cOSD probe sequences for Human RNase P and SARS-CoV-2 are shown in Table 5.

OSD probes have been previously described in the literature in the context of fluorescence reactions. Strand exchange upon the interaction of an OSD probe with a target amplicon generates a fluorescent signal by separating a fluorescent moiety from a quencher molecule. Since an OSD probe targets one of the two exposed single stranded loop regions of the LAMP amplicon, conventionally, the corresponding loop primer is left out of the reaction. Therefore, the standard method for applying OSD probes involves the use of only 5 RT-LAMP primers (FIP, BIP, F3, B3, either LF or LB). Replacing a loop primer with an OSD probe poses a risk as loop primers are accelerants; removal of a loop primers is known to impact the speed and sensitivity of RT-LAMP reactions.

Here, the addition of the 6th LAMP loop primer to amplification mixtures comprising cOSD probes was investigated. If the concentration of the 6th primer is too low, it can negatively impact the reaction performance as stated, but if the concentration is too high, it may negatively impact strand exchange and this LFA signal generation. The later effect is because this loop primer possesses some complementary to the cOSD-A-S sequence. In a preliminary experiment using a different OSD probes with fluorescent readout, it was observed that adding partial levels of the 6th loop primer can improve kinetics, in some cases up to 30%. In a second experiment, it was observed that the sensitivity of cOSD reactions with both Human RNase P-LB-cOSD4 and SARS-CoV-2-LB-cOSD2 probes were improved by partially adding the target loop primers to a concentration of 50% (FIG. 5). The impact of the 6th primer concentration on reaction sensitivity using SARS-CoV-2 cOSD reaction as a model was also investigated.

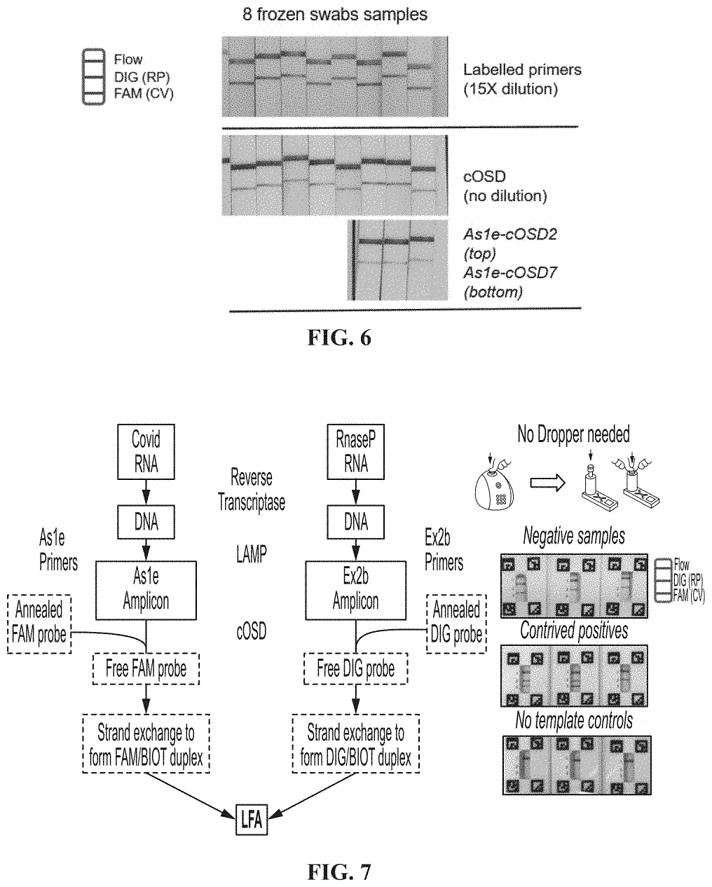

To practically apply cOSD probes in a diagnostic test kit, one would need to rule-out potential false positive results resulting from the interaction of genomic material in the sample and the cOSD probes. In order to de-risk the appearance of false positives, as well as to evaluate the sensitivity of the Human RNase P primers and probes, selected cOSD probe candidates were tested on material from frozen nasal swabs, a model sample type for a SARS-CoV-2 diagnostic kit. As can be seen from FIG. 6, detection of human control was equal between the conventional approach with labelled primers and post-amplification dilution prior to LFA, and a cOSD probes strategy with no post-amplification dilution prior to LFA. Furthermore, false positive activation of the SARS-CoV-2 cOSD probes was not observed.

In order to evaluate the practical application of cOSD probes for the direct application of RT-LAMP products to LFA strips for the multiplexed detection of SARS-CoV-2 and human RNAse P, a small study was conducted using heat-inactivated SARS-CoV-2 virus at 5,000 copies/swab spiked into pooled nasal matrix to generate a contrived positive sample. A schematic depicting the study strategy is shown in FIG. 7. In a preliminary, small scale experiment, the cOSD test kit detected SARS-CoV-2 in all contrived positives, the human RNAse P control in all contrived negatives, and did not report any targets in the No Template Controls (NTCs) (FIG. 7).

The performance of the diagnostic cOSD test kit was further evaluated using actual fresh nasal swabs from donors that were confirmed to be negative for SARS-CoV-2. As presented in Table 1, the test kit detected the human control correctly in 97% of the cases, and no Covid false positives were observed. Furthermore, RNaseP LFA band intensities were above 8 LUMOs Intensity Units, beyond the minimum intensity needed for facile band interpretation. Taken together these results indicate that diagnostic test kits containing cOSD probes can detect positive and negative samples with high specificity with no intervening steps between amplification and application to a LFA.

TABLE 1

| | Human control | Human control | Covid |
|---|---|---|---|
| | True positives | False negatives | false positives |
| Count | 68 | 2 | 0 |
| Percentage [%] | 97 | 3 | 0 |
| 95% CI (Clopper- | 90.0-99.6% | 0.4-9.9% | 0.0-5.0% |
| Pearson) | | | |

### Example 3

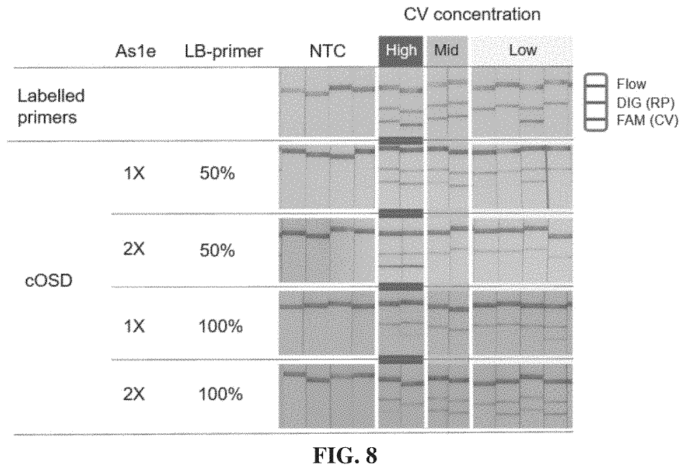

The sensitivity of the cOSD probe system was evaluated. A study investigating COVID template detection sensitivity was performed with varied concentrations of target loop primers and whole primer sets. FIG. 8 shows representative data indicating band intensity and sensitivity are altered by varying concentrations of primers, probes, and template. It was observed that in the context of high or medium COVID template and equal SARS-CoV-2 cOSD probe concentrations, adding more LB loop primers resulted in a reduction of COVID (CV) bands (e.g., as shown in “1×SARS-CoV-2 and 50% LB” vs “1×SARS-CoV-2 and 100% LB” in FIG. 8). Thus, adding target loop primers decreases band intensity—most likely due to complementary binding of the loop primer with the cOSD-A-S oligo. Increasing both primer and probe concentrations results in restoration of COVID detection sensitivity, particularly at medium and low COVID template concentrations (e.g., as shown in “2×SARS-CoV-2 and 100% LB” in FIG. 8).

### Experimental Protocols

Unless stated otherwise, all experiments were carried out by mixing a buffer twice as concentrated (2× buffer-pH 8.07 at 25° C.), either a no primer lyo-bead or a frozen “cold-mix” carrying all enzymes and nucleotides. Primers and RNA templates were prepared in water and mixed subsequently. Probes were prepared in buffer, using labelled oligos at 2.5-5 uM concentration, and annealed on a PCR machine following a 95° C. heating step for 5 mins, cooldown to 25° C. at 0.1° C./s, and hold at 25° C.

TABLE 2
Exemplary Buffer Mix

| Component | Concentration Range | |
|---|---|---|
| Tris-base | 10-25 | mM |
| (NH₄)₂SO₄ | 5-15 | mM |
| Tween ® 20 | 0.05-0.15% | |
| KCl | 40-60 | mM |
| MgSO₄ | 5-10 | mM |
| EGTA | 150-250 | uM |
| dATP | 0.8-1.6 | mM |
| dCTP | 0.8-1.6 | mM |
| dGTP | 0.8-1.6 | mM |
| dTTP | 0.3-0.9 | mM |
| duTP | 0.3-0.9 | mM |
| UDG - Thermolabile | 0.01-0.04 | U/ul |
| RNase inhibitor | 0.2-0.7 | U/ul |
| Reverse Transcriptase enzyme | 0.1-0.5 | U/ul |
| Polymerase | 0.15-0.5 | U/ul |

TABLE 3
Representative Human RNase P Primer Set

| | Concentration | |
|---|---|---|
| Name | [nM] | Sequence [5′-3′] |
| Human | 640 | TGTCGGTCTCTGGCTCCAC |
| RNase P- | | AGGTGGCTGCCAATAC |
| FIP | | (SEQ ID NO: 1) |
| Human | 640 | CACGGGAGCCACTGACTCC |
| RNase P- | | CCTGAAGACTCGGATGT |
| BIP | | (SEQ ID NO: 2) |
| Human | 80 | GCAGCTTCGGGTCCT |
| RNase P- | | (SEQ ID NO: 3) |
| F3 | | |
| Human | 80 | GGATAAGTGGAGGAGTG |
| RNase P- | | TCT |
| B3 | | (SEQ ID NO: 4) |
| Human | 160 | /5DigN/TCAACAAGCTC |
| RNase P- | | CACGGTG |
| LF-DIG | | (SEQ ID NO: 5) |
| Human | 160 | /5Biosg/TCCGCAACA |
| RNase P- | | ACTCAGCC |
| LB-BIO | | (SEQ ID NO: 6) |
| Human | 160 | TCAACAAGCTCCACGGTG |
| RNase P- | | (SEQ ID NO: 7) |
| LF | | |
| Human | 160 | TCCGCAACAACTCAGCC |
| RNase P- | | (SEQ ID NO: 8) |
| LB | | |

TABLE 4
Representative SARS-COV-2 Primer Set

| | Concentration | |
|---|---|---|
| Name | [nM] | Sequence [5′-3′] |
| SAR | 1600 | TCAGCACACAAAGCCAAAAAT |
| S- | | TTATTTTTCTGTGCAAAGGA |
| CoV- | | AATTAAGGAG |
| 2-FIP | | (SEQ ID NO: 9) |
| SAR | 1600 | TATTGGTGGAGCTAAACTTAA |
| S | | AGCCTTTTCTGTACAATCCCT |
| CoV- | | TTGAGTG |
| 2-BIP | | (SEQ ID NO: 10) |
| SAR | 200 | CGGTGGACAAATTGTCAC |
| S- | | (SEQ ID NO: 11) |
| CoV- | | |
| 2-F3 | | |
| SAR | 200 | CTTCTCTGGATTTAACACACTT |
| S- | | (SEQ ID NO: 12) |
| CoV- | | |
| 2-B3 | | |
| SAR | 400 | /56-FAM/TTACAAGCTTAAAGA |
| S | | ATGTCTGAACACT |
| CoV- | | (SEQ ID NO: 13) |
| 2-LF- | | |
| DIG | | |
| SAR | 400 | /5Biosg/TTGAATTTAGGTGAA |
| S | | ACATTTGTCACG |
| CoV- | | (SEQ ID NO: 14) |
| 2- | | |
| LB- | | |
| BIO | | |
| SAR | 400 | TTACAAGCTTAAAGAATGTCTGAA |
| S- | | CACT |
| CoV- | | (SEQ ID NO: 15) |
| 2-LF | | |
| SAR | 400 | TTGAATTTAGGTGAAACATTTGTC |
| S- | | ACG (SEQ ID NO: 16) |
| CoV- | | |
| 2-LB | | |

TABLE 5
Representative cOSD probes for SARS-COV-2 and Human RNase P

| Name | Sequence [5′-3′] |
|---|---|
| SARS-COV- | gggggggaTTGAATTTAGGTGAAACATTTGTCACGCAC/3Invd |
| 2_Blp_cOSD2_A_L | T/ (SEQ ID NO: 17) |
| SARS-COV- | GggggggaTTGAATTTAGGTGAAACATTTGTCACGCAC (SEQ |
| 2_Blp_Cosd2_A_L_ | ID NO: 18) |
| prim | |
| SARS-COV- | GTTTCACCTAAATTCAAtccccccc/36-FAM/ (SEQ ID NO: 19) |
| 2_Blp_cOSD2_A_S_ | |
| FAM | |
| SARS-COV- | gggggggaTTGAATTTAGGTGgccccg/3Bio/ |
| 2_Blp_cOSD2_B_L_ | (SEQ ID NO: 20) |
| BIO | |
| SARS-COV- | cggggcCACCTAAATTCAA/3InvdT/ (SEQ ID NO: 21) |
| 2_Blp_cOSD2_B_S | |
| SARS-COV- | accccgtgcTTGAATTTAGGTGAAACATTTGTCACGCA/3InvdT/ |
| 2_Blp_cOSD3_A_L | (SEQ ID NO: 22) |
| SARS-COV- | AccccgtgcTTGAATTTAGGTGAAACATTTGTCACGCA (SEQ |
| 2_Blp_Cosd3_A_L_ | ID NO: 23) |
| prim | |
| SARS-COV- | TTTCACCTAAATTCAAgcacggggt/36-FAM/ (SEQ ID NO: 24) |
| 2_Blp_cOSD3_A_S_ | |
| FAM | |
| SARS-COV- | ccccgtgcTTGAATTTAGGagggcctgg/3Bio/ (SEQ ID NO: 25) |
| 2_Blp_cOSD3_B_L_ | |
| BIo | |
| SARS-COV- | ccaggccctCCTAAATTCAA/3InvdT/ (SEQ ID NO: 26) |
| 2_Blp_cOSD3_B_S | |
| SARS-COV- | TTGAATTTAGGTGAAACATTTGTCACGCACgtccacc/3InvdT/ |
| 2_Blp_cOSD4_A_L | (SEQ ID NO: 27) |
| SARS-COV- | TTGAATTTAGGTGAAACATTTGTCACGCACgtccacc (SEQ |
| 2_Blp_cOSD4_A_L_ | ID NO: 28) |
| prim | |
| SARS-COV- | ggtggacGTGCGTGACAAATGTTT/36-FAM/ (SEQ ID NO: 29) |
| 2_Blp_cOSD4_A_S_ | |
| FAM | |
| SARS-COV- | cccgTTTGTCACGCACgtccac/3Bio/ (SEQ ID NO: 30) |
| 2_Blp_cOSD4_B_L_ | |
| BIO | |
| SARS-COV- | GTGCGTGACAAAcggg/3InvdT/ (SEQ ID NO: 31) |
| 2_Blp_cOSD4_B_S | |
| SARS-COV- | TTGAATTTAGGTGAAACATTTGTCACGCaaccgcgaat/3InvdT/ |
| 2_Blp_cOSD5_A_L | (SEQ ID NO: 32) |
| SARS-COV- | TTGAATTTAGGTGAAACATTTGTCACGCaaccgcgaat (SEQ |
| 2_Blp_cOSD5_A_L_ | ID NO: 33) |
| prim | |
| SARS-COV- | attcgcggttGCGTGACAAATGTTT/36-FAM/ (SEQ ID NO: 34) |
| 2_Blp_cOSD5_A_S_ | |
| FAM | |
| SARS-COV- | tcgcgtTTTGTCACGCaaccgcga/3Bio/ (SEQ ID NO: 35) |
| 2_Blp_cOSD5_B_L_ | |
| BIO | |
| SARS-COV- | GCGTGACAAAacgcga/3InvdT/ (SEQ ID NO: 36) |
| 2_Blp_cOSD5_B_S | |
| SARS-COV- | aggccgcttgTTGAATTTAGGTGAAACATTTGTCACGCA/3Invd |
| 2_Blp_cOSD6_A_L | T/ (SEQ ID NO: 37) |
| SARS-COV- | AggccgcttgTTGAATTTAGGTGAAACATTTGTCACGCA (SEQ |
| 2_Blp_cOSD6_A_L_ | ID NO: 38) |
| prim | |
| SARS-COV- | GTTTCACCTAAATTCAAcaagcggcct/36-FAM/ (SEQ ID NO: |
| 2_Blp_cOSD6_A_S_ | 39) |
| FAM | |
| SARS-COV- | ggccgcttgTTGAATTTAGGTggaggccc/3Bio/ |
| 2_Blp_cOSD6_B_L_ | (SEQ ID NO: 40) |
| BIO | |
| SARS-COV- | gggcctccACCTAAATTCAA/3InvdT/ (SEQ ID NO: 41) |
| 2_Blp_cOSD6_B_S | |
| SARS-COV- | TTGAATTTAGGTGAAACATTTGTCACGCAcagcctctagt/3Invd |
| 2_Blp_cOSD7_A_L | T/ (SEQ ID NO: 42) |
| SARS-COV- | TTGAATTTAGGTGAAACATTTGTCACGCAcagcctctagt (SEQ |
| 2_Blp_Cosd7_A_ | ID NO: 43) |
| L_prim | |
| SARS-COV- | actagaggctgTGCGTGACAAATGTT/36-FAM/ (SEQ ID NO: 44) |
| 2_Blp_cOSD7_A_S_ | |
| FAM | |
| SARS-COV- | tgcgtggTGTCACGCAcagcctctag/3Bio/ (SEQ ID NO: 45) |
| 2_Blp_cOSD7_B_L_ | |
| BIo | |
| SARS-COV- | TGCGTGACAccacgca/3InvdT/ (SEQ ID NO: 46) |
| 2_Blp_cOSD7_B_S | |
| SARS-COV- | TTGAATTTAGGTGAAACATTTGTCACGCgctctctata/3InvdT/ |
| 2_Blp_cOSD8_A_L | (SEQ ID NO: 47) |
| SARS-COV- | TTGAATTTAGGTGAAACATTTGTCACGCgctctctata (SEQ ID |
| 2_Blp_Cosd8_A_L_ | NO: 48) |
| prim | |
| SARS-COV- | tatagagagcGCGTGACAAATGTT/36-FAM/ (SEQ ID NO: 49) |
| 2_Blp_cOSD8_A_S_ | |
| FAM | |
| SARS-COV- | agcgccTGTCACGCgctctct/3Bio/ (SEQ ID NO: 50) |
| 2_Blp_cOSD8_B_L_ | |
| BIO | |
| SARS-COV- | GCGTGACAggcgct/3InvdT/ (SEQ ID NO: 51) |
| 2_Blp_cOSD8_B_S | |
| SARS-COV- | TTGAATTTAGGTGAAACATTTGTCACGCActcacgact/3InvdT/ |
| 2_Blp_cOSD9_A_L | (SEQ ID NO: 52) |
| SARS-COV- | TTGAATTTAGGTGAAACATTTGTCACGCActcacgact (SEQ |
| 2_Blp_cOSD9_A_L_ | ID NO: 53) |
| prim | |
| SARS-COV- | agtcgtgagTGCGTGACAAATGTT/36-FAM/ |
| 2_Blp_cOSD9_A_S_ | (SEQ ID NO: 54) |
| FAM | |
| SARS-COV- | cgcggTGTCACGCActcacg/3Bio/ (SEQ ID NO: 55) |
| 2_Blp_cOSD9_B_L_ | |
| BIO | |
| SARS-COV- | TGCGTGACAccgcg/3InvdT/ (SEQ ID NO: 56) |
| 2_Blp_cOSD9_B_S | |
| SARS-COV- | TTACAAGCTTAAAGAATGTCTGAACACTcgggtctatt/3InvdT/ |
| 2_Flp_cOSD1_A_L | (SEQ ID NO: 57) |
| SARS-COV- | TTACAAGCTTAAAGAATGTCTGAACACTcgggtctatt (SEQ ID |
| 2_Flp_Cosd1_A_L_ | NO: 58) |
| prim | |
| SARS-COV- | aatagacccgAGTGTTCAGACATTC/36-FAM/ (SEQ ID NO: 59) |
| 2_Flp_cOSD1_A_S_ | |
| FAM | |
| SARS-COV- | acgatgcGTCTGAACACTcgggtctat/3Bio/ (SEQ ID NO: 60) |
| 2_Flp_cOSD1_B_L_ | |
| BIO | |
| SARS-COV- | AGTGTTCAGACgcatcgt/3InvdT/ (SEQ ID NO: 61) |
| 2_Flp_cOSD1_B_S | |
| SARS-COV- | acacggogTTACAAGCTTAAAGAATGTCTGAACACTCTCC/3I |
| 2_Flp_cOSD2_A_L | nvdT/ (SEQ ID NO: 62) |
| SARS-COV- | AcacggcgTTACAAGCTTAAAGAATGTCTGAACACTCTCC |
| 2_Flp_cOSD2_A_L_ | (SEQ ID NO: 63) |
| prim | |
| SARS-COV- | ACATTCTTTAAGCTTGTAAcgccgtgt/36-FAM/ (SEQ ID NO: |
| 2_Flp_cOSD2_A_S_ | 64) |
| FAM | |
| SARS-COV- | acggcgTTACAAGCTTAAAaaagcagat/3Bio/ (SEQ ID NO: 65) |
| 2_Flp_cOSD2_B_L_ | |
| BIO | |
| SARS-COV- | atctgctttTTTAAGCTTGTAA/3InvdT/ (SEQ ID NO: 66) |
| 2_Flp_cOSD2_B_S | |
| Human RNase | taaatagccgGGATCCGCAACAACTCAGCCATCCA/ |
| P_Blp_cOSD1_A_L | 3InvdT/ (SEQ ID NO: 67) |
| Human RNase | TaaatagccgGGATCCGCAACAACTCAGCCATCCA (SEQ ID |
| P_Blp_cOSD1_A_L_ | NO: 68) |
| prim | |
| Human RNase | TTGTTGCGGATCCcggctattta/3Dig_N/ (SEQ ID NO: 69) |
| P_Blp_cOSD1_A_S_ | |
| DIG | |
| Human RNase | TTGTTGCGGATCCcggctattta/36-FAM/ (SEQ ID NO: 70) |
| P_Blp_cOSD1_A_S_ | |
| FAM | |
| Human RNase | aaatagccgGGATCCGCcggacgtg/3Bio/ (SEQ ID NO: 71) |
| P_Blp_cOSD1_B_L_ | |
| BIO | |
| Human RNase | cacgtccgGCGGATCC/3InvdT/ (SEQ ID NO: 72) |
| P_Blp_cOSD1_B_S | |
| Human RNase | GGATCCGCAACAACTCAGCCATCCcagtccttat/3InvdT/ (SEQ |
| P_Blp_cOSD2_A_L | ID NO: 73) |
| Human RNase | GGATCCGCAACAACTCAGCCATCCcagtccttat (SEQ ID NO: |
| P_Blp_Cosd2_A_L_ | 74) |
| prim | |
| Human RNase | ataaggactgGGATGGCTGAGT/3Dig_N/ (SEQ ID NO: 75) |
| P_Blp_cOSD2_A_S_ | |
| DIG | |
| Human RNase | ataaggactgGGATGGCTGAGT/36-FAM/ (SEQ ID NO: 76) |
| P_Blp_cOSD2_A_S_ | |
| FAM | |
| Human RNase | tcggcCTCAGCCATCCcagtcctta/3Bio/ (SEQ ID NO: 77) |
| P_Blp_cOSD2_B_L_ | |
| BIO | |
| Human RNase | GGATGGCTGAGgccga/3InvdT/ (SEQ ID NO: 78) |
| P_Blp_cOSD2_B_S | |
| Human RNase | GGATCCGCAACAACTCAGCCATCCACAccgtgcag/3InvdT/ |
| P_Blp_cOSD4_A_L | (SEQ ID NO: 79) |
| Human RNase | GGATCCGCAACAACTCAGCCATCCACAccgtgcag (SEQ ID |
| P_Blp_Cosd4_A_L_ | NO: 80) |
| prim | |
| Human RNase | ctgcacggTGTGGATGGCTGAGT/3Dig_N/ (SEQ ID NO: 81) |
| P_Blp_cOSD4_A_S_ | |
| DIG | |
| Human RNase | ctgcacggTGTGGATGGCTGAGT/36-FAM/ (SEQ ID NO: 82) |
| P_Blp_cOSD4_A_S_ | |
| FAM | |
| Human RNase | tctgcggaCCATCCACAccgtgca/3Bio/ (SEQ ID NO: 83) |
| P_Blp_cOSD4_B_L_ | |
| BIO | |
| Human RNase | TGTGGATGGtccgcaga/3InvdT/ (SEQ ID NO: 84) |
| P_Blp_cOSD4_B_S | |
| Human RNase | GCTCATCAACAAGCTCCACGGTGGAGGtacaaaggta/3InvdT/ |
| P_Flp_cOSD1_A_L | (SEQ ID NO: 85) |
| Human RNase | GCTCATCAACAAGCTCCACGGTGGAGGtacaaaggta (SEQ ID |
| P_Flp_Cosd1_A_L_ | NO: 86) |
| prim | |
| Human RNase | tacctttgtaCCTCCACCGTGGAGC/3Dig_N/ (SEQ ID NO: 87) |
| P_Flp_cOSD1_A_S_ | |
| DIG | |
| Human RNase | tacctttgtaCCTCCACCGTGGAGC/36-FAM/ (SEQ ID NO: 88) |
| P_Flp_cOSD1_A_S_ | |
| FAM | |
| Human RNase | aacggtaACGGTGGAGGtacaaaggt/3Bio/ (SEQ ID NO: 89) |
| P_Flp_cOSD1_B_L_ | |
| BIO | |
| Human RNase | CCTCCACCGTtaccgtt/3InvdT/ (SEQ ID NO: 90) |
| P_Flp_cOSD1_B_S | |

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An amplification reaction probe set comprising:
(i) a first hemi-duplex polynucleotide comprising a first polynucleotide strand hybridized to a second polynucleotide strand to form a duplex portion, each polynucleotide strand having a 5′ end and a 3′ end, the 5′ end of the first polynucleotide forming a target sequence-specific single stranded portion that extends past the 3′ end of the second polynucleotide strand, and the 3′ end of the second polynucleotide strand comprising a first label; and,
(ii) a second hemi-duplex polynucleotide comprising a third polynucleotide strand hybridized to a fourth polynucleotide strand to form a duplex portion, each polynucleotide strand having a 5′ end and a 3′ end, the 3′ end of the third polynucleotide strand forming a single stranded portion having a region of complementarity to the duplex portion of the second polynucleotide strand of the first hemi-duplex polynucleotide and comprising a second label.

2. The amplification reaction probe set of claim 1, wherein
(i) the first polynucleotide strand and the second polynucleotide strand each independently range in length from about 10 nucleotides to about 50 nucleotides; and/or
(ii) the third polynucleotide strand and the fourth polynucleotide strand each independently range in length from about 10 nucleotides to about 50 nucleotides.

3. The amplification reaction probe set of claim 1, wherein the duplex region of the first hemi-duplex polynucleotide comprises a blunt end or wherein the duplex region of the second hemi-duplex polynucleotide comprises a blunt end.

4. The amplification reaction probe set of claim 1, wherein
(i) the duplex region of the first hemi-duplex polynucleotide comprises a GC clamp, optionally where the GC clamp ranges in length from 1 nucleotide to about 10 nucleotides, optionally wherein the GC clamp is 5 or 6 nucleotides in length; and/or
(ii) the duplex region of the second hemi-duplex polynucleotide comprises a GC clamp, optionally where the GC clamp ranges in length from 1 nucleotide to about 10 nucleotides, further optionally wherein the GC clamp is 5 or 6 nucleotides in length.

5. The amplification reaction probe set of claim 1, wherein the duplex region of the first hemi-duplex polynucleotide comprises one or more branch migration domains and/or the duplex region of the second hemi-duplex polynucleotide comprises one or more branch migration domains.

6. The amplification reaction probe set of claim 1, wherein the single stranded portion of the first hemi-duplex polynucleotide is at least three nucleotides longer than the duplex portion of the first hemi-duplex polynucleotide, or wherein the single stranded portion of the second hemi-duplex polynucleotide is the same length as the duplex portion of the second hemi-duplex polynucleotide.

7. The amplification reaction probe set of claim 1, wherein the target sequence-specific single stranded portion has a region of complementarity with a target polynucleotide, wherein the target polynucleotide is a Loop-mediated isothermal amplification (LAMP) amplicon.

8. The amplification reaction probe set of claim 7, wherein the duplex portion of the second hemi-duplex polynucleotide does not have a region of complementarity with the target polynucleotide.

9. The amplification reaction probe set of claim 1, wherein the first label is selected from FAM, FITC, digoxigenin (DIG), dinitrophenyl (DNP), and biotin, and/or the second label is selected from FAM, FITC, digoxigenin (DIG), dinitrophenyl (DNP), and biotin.

10. A dual-labeled molecule comprising the second polynucleotide strand of claim 1 hybridized to the third polynucleotide strand of claim 1.

11. An amplification mixture comprising:
(i) the amplification reaction probe set of claim 1;
(ii) one or more buffering agents, one or more salts, and, optionally, one or more detergents;
(iii) a deoxynucleoside triphosphate (dNTP) mixture;
(iv) a polymerase; and, optionally,
(v) a reverse transcriptase.

12. A method for producing dual-labeled detection products, the method comprising:
(a) performing an isothermal amplification reaction to amplify a target nucleic acid in the presence of:
(i) a first hemi-duplex polynucleotide comprising a first polynucleotide strand hybridized to a second polynucleotide strand to form a duplex portion, each polynucleotide strand having a 5′ end and a 3′ end, the 5′ end of the first polynucleotide forming a target nucleic acid sequence-specific single stranded portion that extends past the 3′ end of the second polynucleotide strand, and the 3′ end of the second polynucleotide strand comprising a first label;
(ii) a second hemi-duplex polynucleotide comprising a third polynucleotide strand hybridized to a fourth polynucleotide strand to form a duplex portion, each polynucleotide strand having a 5′ end and a 3′ end, the 3′ end of the third polynucleotide strand forming a single stranded portion having a region of complementarity to the duplex portion of the second polynucleotide strand of the first hemi-duplex polynucleotide and comprising a second label;
(iii) one or more additional primers that bind to the target nucleic acid;
(iv) a deoxynucleoside triphosphate (dNTP) mixture;
(v) a polymerase; and, optionally,
(vi) a reverse transcriptase; and
(b) producing dual-labeled detection products by using an oligonucleotide strand displacement reaction to form a duplex molecule comprising the second polynucleotide strand of (i) and the third polynucleotide strand of (ii).

13. The method of claim 12, wherein the isothermal amplification reaction is loop-mediated isothermal amplification (LAMP).

14. The method of claim 12, wherein the one more additional primers comprises 2, 3, 4, 5, or 6 additional primers.

15. The method of claim 12, wherein the one or more additional primers are LAMP primers.

16. The method of claim 12, wherein the first hemi-duplex polynucleotide is a first hemi-duplex polynucleotide of an amplification reaction probe set.

17. The method of claim 12, wherein the second hemi-duplex polynucleotide is a second hemi-duplex polynucleotide of an amplification reaction probe set.

18. The method of claim 12, wherein the target nucleic acid is derived from a pathogen or derived from a human.

19. A method for indirectly detecting isothermal amplification of a target nucleic acid in a reaction, the method comprising:
producing one or more dual-labeled detection products according to the method of claim 12; and
contacting a lateral flow assay (LFA) device with the one or more dual-labeled detection products to produce one or more detectable signals; and
identifying the presence of the target nucleic acid in the isothermal amplification reaction based upon detecting the presence of the detectable signal produced by the one or more dual-labeled detection products.

20. The method of claim 19, wherein the target nucleic acid is derived from a pathogen or derived from a human.
